(19) United States
(12) Patent Application Publication
(10) Pub. No.: US 2025/0011591 A1
(43) Pub. Date: Jan. 9, 2025

(54) Sustainable, High Flow Liquid Crystalline Polymer Composition

(71) Applicant: Ticona LLC, Florence, KY (US)

(72) Inventor: Young Shin Kim, Cincinnati, OH (US)

(21) Appl. No.: 18/746,310

(22) Filed: Jun. 18, 2024

Related U.S. Application Data
(60) Provisional application No. 63/510,649, filed on Jun. 28, 2023.

(51) Int. Cl. C08L 67/04; C07C 51/15; H01R 13/533

(52) U.S. Cl. CPC C08L 67/04; C07C 51/15; C08L 2201/02; C08L 2203/20; C08L 2205/025; C08L 2205/03; H01R 13/533

(57) ABSTRACT

A polymer composition that comprises a polymer matrix containing a first liquid crystalline polymer that includes one or more monomers derived from bio-naphtha is provided. The polymer composition exhibits a melt viscosity of about 60 Pa-s or less as determined in accordance with ISO 11443:2021 at a shear rate of 1,000 seconds⁻¹ and temperature that is 15° C. higher than the melting temperature of the composition.

### RELATED APPLICATION

The present application is based upon and claims priority to U.S. Provisional Patent Application Ser. No. 63/510,649, having a filing date of Jun. 28, 2023, which is incorporated herein by reference.

### BACKGROUND OF THE INVENTION

Electrical components (e.g., fine pitch connectors) are commonly produced from wholly aromatic thermotropic liquid crystalline polymers (“LCPs”). One benefit of such polymers is that they can exhibit a relatively high “flow”, which refers to the ability of the polymer when heated under shear to uniformly fill complex parts at fast rates without excessive flashing or other detrimental processing issues. In addition to enabling complex part geometries, high polymer flow can also enhance the ultimate performance of the molded component. Most-notably, parts generated from well-flowing polymers generally display improved dimensional stability owing to the lower molded-in stress, which makes the component more amenable to downstream thermal processes that can be negatively impacted from warpage and other polymer stress relaxation processes that occur in less well-molded materials. Despite their benefits, current commercial “high flow” LCP compositions tend to be formed from raw materials that have been produced from crude oil through a catalytic cracking process. Recently, however, a need for a more carbon neutral approach has been sought. To be carbon neutral, a company must remove the same amount of carbon dioxide that it is emitting into the atmosphere to achieve a net-zero carbon emissions. A carbon negative company, on the other hand, removes more carbon from the atmosphere than it releases. In view of the significant efforts across the globe of companies to become carbon neutral or carbon negative, a need exists for liquid crystalline polymer compositions that are more sustainable that yet are able to maintain high flow properties.

### SUMMARY OF THE INVENTION

In accordance with one embodiment of the present invention, a polymer composition is disclosed that comprises a polymer matrix containing a first liquid crystalline polymer that includes one or more monomers derived from bio-naphtha. The polymer composition exhibits a melt viscosity of about 60 Pa-s or less as determined in accordance with ISO 11443:2021 at a shear rate of 1,000 seconds⁻¹ and temperature that is 15° C. higher than the melting temperature of the composition.

Other features and aspects of the present invention are set forth in greater detail below.

### BRIEF DESCRIPTION OF THE FIGURES

A full and enabling disclosure of the present invention, including the best mode thereof to one skilled in the art, is set forth more particularly in the remainder of the specification, including reference to the accompanying figures, in which:

FIG. 1A depicts a thin walled electrical connector according to aspects of present invention;

FIG. 1B depicts an enlarged view of a portion of thin walled connector of FIG. 1A; and

FIG. 2 is an exploded perspective view of another embodiment of a thin walled connector and connector receptacle that may be formed according to the present invention.

### DETAILED DESCRIPTION

It is to be understood by one of ordinary skill in the art that the present discussion is a description of exemplary embodiments only, and is not intended as limiting the broader aspects of the present invention.

Generally speaking, the present invention is directed to a polymer composition that contains a polymer matrix containing bio-LCP, which includes one or more monomers derived from bio-naphtha. Through careful control over the specific nature and concentration of the components employed in the composition, the present inventor has discovered that the resulting composition can be formed that has a unique combination of having a certain bio-content and yet still maintaining a low melt viscosity. For example, the bio-LCP may have a “bio-content”of about 5 wt. % to 100 wt. %, in some embodiments from about 10 wt. % to about 90 wt. %, in some embodiments from about 20 wt. % to about 70 wt. %, and in some embodiments, from about 30 wt. % to about 60 wt. % based on the total weight of monomers (repeating units) employed in the polymer. As used herein, the term “bio-content” generally refers to the weight percentage of monomers (repeating units) that are derived from bio-naphtha. Thus, it should be understood that this weight percentage may include the bio-hydroxybenzoic acids described herein (e.g., bio-4-hydroxybenzoic acid), as well as other monomer components that may also be derived from bio-naphtha, such as bio-terephthalic acid (“bio-TA”), bio-isophthalic acid (“bio-IA”), bio-4,4′-biphenol (“bio-BP”), bio-hydroquinone (“bio-HQ”), bio-2-hydroxy-6-naphthoic acid (“bio-HNA”), bio-2,6-naphthalenedicarboxylic acid (“bio-NDA”), bio-4-aminophenol (“bio-AP”), bio-acetaminophen (“bio-APAP”), etc. If desired, other additives employed in the polymer composition may also be derived from a sustainable source, such as recycled materials, renewable materials, bio-based materials, etc. For example, the total “sustainable content” of the polymer composition is typically from about 10 wt. % to 100 wt. %, in some embodiments from about 20 wt. % to about 90 wt. %, and in some embodiments, from about 30 wt. % to about 80 wt. % based on the total weight of the composition. The term “sustainable content” generally refers to the weight percentage of components that are derived from a sustainable source. For a composition containing only bio-LCP, for example, the “sustainable content” is the same as the “bio-content” (weight percentage of monomers derived from bio-naphtha). For compositions containing bio-LCP, other sustainable materials (e.g., recycled materials), non-sustainable materials (e.g., fossil fuel-based materials, virgin materials, etc.), the “sustainable content” can be determined as follows:

(Weight of Bio-LCP)*(“Bio-Content”)+(Weight of Other Sustainable Materials)/Weight of Polymer Composition

Despite containing bio-LCP, the resulting polymer composition may still exhibit a low melt viscosity, such as about 60 Pa-s or less, in some embodiments about 50 Pa-s or less, in some embodiments about 30 Pa-s or less, in some embodiments from about 1 to about 25 Pa-s, in some embodiments from about 2 about 20 Pa-s, in some embodiments from about 3 to about 15 Pa-s, and in some embodiments, from about 4 to about 12 Pa-s, as determined at a shear rate of 1,000 seconds⁻¹ in accordance with ISO 11443:2021 at a temperature that is 15° C. higher than the melting temperature of the composition (e.g., about 360° C. for a melting temperature of about 345° C.).

The polymer composition may also exhibit a high melting temperature, such as about 300° C. or more, in some embodiments from about 320° C. to about 400° C., in some embodiments from about 330° C. to about 380° C., and in some embodiments, from about 340° C. to about 370° C. Even at such melting temperatures, the ratio of the deflection temperature under load (“DTUL”), a measure of short term heat resistance, to the melting temperature may still remain relatively high. For example, the ratio may range from about 0.5 to about 1.00, in some embodiments from about 0.6 to about 0.95, and in some embodiments, from about 0.65 to about 0.85. The specific DTUL values may, for instance, be 170° C. or more, in some embodiments about 200° C. or more, in some embodiments about 220° C. or more, in some embodiments about 230° C. or more, in some embodiments from about 240° C. to about 300° C., and in some embodiments, from about 250° C. to about 280° C. Such high DTUL values can, among other things, allow the use of high speed and reliable surface mounting processes for mating the structure with other components of the electrical component. The heat resistance of the polymer composition may also be reflected by the “blister-free temperature”, as described in more detail below, which may be about 240° C. or more, in some embodiments about 250° C. or more, in some embodiments from about 260° C. to about 300° C., and in some embodiments, from about 270° C. to about 290° C.

The polymer composition may also exhibit excellent mechanical properties. For example, the composition may exhibit a relatively high tensile and/or flexural modulus, which allows it to remain relatively stiff once molded into a thin part. The tensile modulus may, for instance, be about 6,000 MPa or more, in some embodiments about 8,000 MPa or more, in some embodiments from about 9,000 MPa to about 16,000 MPa, and in some embodiments, from about 10,000 MPa to about 15,000 MPa as determined in accordance with ISO 527:2019 at a temperature of 23° C. The flexural modulus may likewise be about 6,000 MPa or more, in some embodiments about 8,000 MPa or more, in some embodiments from about 9,000 MPa to about 16,000 MPa, and in some embodiments, from about 10,000 MPa to about 15,000 MPa as determined in accordance with ISO 178:2019 at a temperature of 23° C. Of course, the polymer composition may also exhibit other good tensile and flexural properties. For example, the polymer composition may exhibit a tensile strength of about 50 MPa or more, in some embodiments from about 60 MPa to about 250 MPa, and in some embodiments, from about 70 MPa to about 150 MPa and/or a tensile elongation of about 0.5% or more, in some embodiments from about 0.6% to about 3.5%, and in some embodiments, from about 0.8% to about 3%, as determined in accordance with ISO 527:2019 at a temperature of 23° C. The polymer composition may also exhibit a flexural strength of about 80 MPa or more, in some embodiments from about 100 MPa to about 350 MPa, and in some embodiments, from about 120 MPa to about 250 MPa and/or a flexural elongation of about 0.5% or more, in some embodiments from about 0.6% to about 3.5%, and in some embodiments, from about 0.8% to about 3%, as determined in accordance with ISO 178:2019 at a temperature of 23° C. The impact strength may also be good. For example, the composition may exhibit a Charpy notched impact strength of about 1 KJ/m² or more, in some embodiments from about 1.5 to about 10 KJ/m², and in some embodiments, from about 2 to about 80 KJ/m², measured at 23° C. according to ISO Test No. 179-1:2010.

The flame retardant properties of the composition may also be good, such as characterized in accordance with the vertical burn test procedure of UL94 of the “Tests for Flammability of Plastic Materials for Parts in Devices and Appliances” (edition date of Feb. 28, 2023). According to this procedure, for example, the composition may exhibit a V-0 rating at a part thickness of about 0.8 millimeters, as well as even smaller thicknesses, such as about 0.4 or about 0.2 millimeters. To achieve a V-0 rating, for example, the polymer composition generally exhibits no drips that ignite the cotton batting. However, the polymer composition may even exhibit less than 3 drips, in some embodiments less than 2 drips, in some embodiments less than 1 drip (e.g., 0 drips) that do not ignite the cotton batting. The polymer composition may also exhibit a total flame time (t1+t2) of about 50 seconds or less, in some embodiments about 40 seconds or less, in some embodiments about 30 seconds or less, and in some embodiments, about 20 seconds or less. The polymer composition may exhibit a V-0 rating before aging (“unaged V-0 rating”) and/or after aging at a temperature of 70° C. for 7 days (“aged V-0 rating”).

Various embodiments of the present invention will now be described in more detail.

### I. Polymer Composition

### A. Polymer Matrix

The polymer matrix typically contains one or more bio-liquid crystalline polymers, generally in an amount of from about 40 wt. % to about 95 wt. %, in some embodiments from about 45 wt. % to about 90 wt. %, and in some embodiments, from about 50 wt. % to about 85 wt. % of the polymer composition. The liquid crystalline polymers are generally classified as “thermotropic” to the extent that they can possess a rod-like structure and exhibit a crystalline behavior in their molten state (e.g., thermotropic nematic state). The polymers may have a relatively high melting temperature, such as about 280° C. or more, in some embodiments about 300° C. or more, in some embodiments from about 320° C. to about 400° C., in some embodiments from about 330° C. to about 380° C., and in some embodiments, from about 340° C. to about 370° C. Such polymers may be formed from one or more types of repeating units as is known in the art. A liquid crystalline polymer may, for example, contain one or more aromatic ester repeating units generally represented by the following Formula (I):

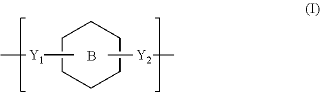

wherein,
ring B is a substituted or unsubstituted 6-membered aryl group (e.g., 1,4-phenylene or 1,3-phenylene), a substituted or unsubstituted 6-membered aryl group fused to a substituted or unsubstituted 5- or 6-membered aryl group (e.g., 2,6-naphthalene), or a substituted or unsubstituted 6-membered aryl group linked to a substituted or unsubstituted 5- or 6-membered aryl group (e.g., 4,4-biphenylene); and
Y₁ and Y₂ are independently O, C(O), NH, C(O)HN, or NHC(O).

Typically, at least one of Y₁ and Y₂ are C(O). Examples of such aromatic ester repeating units may include, for instance, aromatic dicarboxylic repeating units (Y₁ and Y₂ in Formula I are C(O)), aromatic hydroxycarboxylic repeating units (Y₁ is O and Y₂ is C(O) in Formula I), as well as various combinations thereof.

Aromatic hydroxycarboxylic repeating units, for instance, may be employed that are derived from aromatic hydroxycarboxylic acids, such as, 4-hydroxybenzoic acid; 4-hydroxy-4′-biphenylcarboxylic acid; 2-hydroxy-6-naphthoic acid; 2-hydroxy-5-naphthoic acid; 3-hydroxy-2-naphthoic acid; 2-hydroxy-3-naphthoic acid; 4′-hydroxyphenyl-4-benzoic acid; 3′-hydroxyphenyl-4-benzoic acid; 4′-hydroxyphenyl-3-benzoic acid, etc., as well as alkyl, alkoxy, aryl and halogen substituents thereof, and combination thereof. Particularly suitable aromatic hydroxycarboxylic acids are 4-hydroxybenzoic acid (“HBA”) and 6-hydroxy-2-naphthoic acid (“HNA”). When employed, repeating units derived from hydroxycarboxylic acids (e.g., HBA and/or HNA) typically constitute about 20 mol. % or more, in some embodiments about 30 mol. % or more, in some embodiments from about 35 mol. % to 85 mol. %, and in some embodiments, from about 40 mol. % to about 70 mol. % of the polymer.

Aromatic dicarboxylic repeating units may also be employed that are derived from aromatic dicarboxylic acids, such as terephthalic acid, isophthalic acid, 2,6-naphthalenedicarboxylic acid, diphenyl ether-4,4′-dicarboxylic acid, 1,6-naphthalenedicarboxylic acid, 2,7-naphthalenedicarboxylic acid, 4,4′-dicarboxybiphenyl, bis(4-carboxyphenyl) ether, bis(4-carboxyphenyl) butane, bis(4-carboxyphenyl) ethane, bis(3-carboxyphenyl) ether, bis(3-carboxyphenyl) ethane, etc., as well as alkyl, alkoxy, aryl and halogen substituents thereof, and combinations thereof. Particularly suitable aromatic dicarboxylic acids may include, for instance, terephthalic acid (“TA”), isophthalic acid (“IA”), and 2,6-naphthalenedicarboxylic acid (“NDA”). When employed, repeating units derived from aromatic dicarboxylic acids (e.g., IA, TA, and/or NDA) each typically constitute from about 5 mol. % to about 40 mol. %, in some embodiments from about 10 mol. % to about 35 mol. %, and in some embodiments, from about 15 mol. % to about 30 mol. % of the polymer.

Other repeating units may also be employed in the polymer. In certain embodiments, for instance, repeating units may be employed that are derived from aromatic diols, such as hydroquinone, resorcinol, 2,6-dihydroxynaphthalene, 2,7-dihydroxynaphthalene, 1,6-dihydroxynaphthalene, 4,4′-dihydroxybiphenyl (or 4,4′-biphenol), 3,3′-dihydroxybiphenyl, 3,4′-dihydroxybiphenyl, 4,4′-dihydroxybiphenyl ether, bis(4-hydroxyphenyl) ethane, etc., as well as alkyl, alkoxy, aryl and halogen substituents thereof, and combinations thereof. Particularly suitable aromatic diols may include, for instance, hydroquinone (“HQ”) and 4,4′-biphenol (“BP”). When employed, repeating units derived from aromatic diols (e.g., HQ and/or BP) typically constitute from about 1 to about 40 mol. %, in some embodiments from about 5 mol. % to about 35 mol. %, and in some embodiments, from about 10 mol. % to about 30 mol. % of the polymer.

Repeating units may also be employed, such as those derived from aromatic amides (e.g., acetaminophen (“APAP”)) and/or aromatic amines (e.g., 4-aminophenol (“AP”), 3-aminophenol, 1,4-phenylenediamine, 1,3-phenylenediamine, etc.). When employed, repeating units derived from aromatic amides (e.g., APAP) and/or aromatic amines (e.g., AP) typically constitute from about 0.1 mol. % to about 20 mol. %, in some embodiments from about 0.5 mol. % to about 15 mol. %, and in some embodiments, from about 1 mol. % to about 10 mol. % of the polymer. It should also be understood that various other monomeric repeating units may be incorporated into the polymer. For instance, in certain embodiments, the polymer may contain one or more repeating units derived from non-aromatic monomers, such as aliphatic or cycloaliphatic hydroxycarboxylic acids, dicarboxylic acids, diols, amides, amines, etc. Of course, in other embodiments, the polymer may be “wholly aromatic” in that it lacks repeating units derived from non-aromatic (e.g., aliphatic or cycloaliphatic) monomers.

As noted above, one or more of the monomeric constituents of the liquid crystalline polymer are derived from “bio-naphtha”, which is generally refers to naphtha produced from a renewable source. Bio-naphtha is a hydrocarbon composition that primarily includes paraffins that can be converted into a bio-aromatic compound (e.g., bio-benzene). The hydrocarbon content typically has from 8 to 24 carbons, and in some cases from 10 to 18 carbons. To form bio-naphtha, a bio-distillate feedstock is typically provided that includes a complex mixture of natural occurring fats and/or oils, such as plant-based fats and oils (e.g., cotton, coconut, corn, palm, peanut, linseed, rice, rapeseed, olive, soybean, sunflower, linola, tallow, tall, castor, etc.) and animal-based fats and oils (e.g., butter or milk fat). The feedstock may be provided in an unused (virgin) state and/or obtained from a waste product, such as cooking oils, inedible highly saturated oils, waste food oils, by-products of the refining of vegetable oils, and mixtures thereof. Natural fats and oils primarily include triglycerides and to some extent of free fatty acids (FFA). Many different types of triglycerides are produced in nature, either from vegetable or from animal origin. Fatty acids in fats & oils are found esterified to glycerol (triacylglycerol). The acyl-group is a long-chain (C₁₂-C₂₂) hydrocarbon with a carboxyl-group at the end that is generally esterified with glycerol. Fats and oils are characterized by the chemical composition and structure of its fatty acid moiety. The fatty acid moiety can be saturated or contain one or more double bonds. The aforementioned sources of sources of fats and/or oils may include fatty acids, such as saturated fatty acids (e.g., caproic, caprylic, capric, lauric, myristic, palmitic, margaric, stearic, arachidic, behenic, lignoceric, etc.) and/or unsaturated fatty acids (e.g., myristoleic, palmitoleic, heptadecenoic, oleic, linoleic, linolenic, gadolenic, riconoleic, rosin, etc.). Tall oil, for example, contains myristic acid, stearic acid, arachidic acid, oleic acid, linoleic acid, and gadolenic acid.

Bio-distillate feedstocks may be classified based on their free fatty acid (FFA) content as follows: refined oils, such as soybean or refined canola oils (FFA<1.5%); low free fatty acid yellow greases and animal fats (FFA<4%); and high free fatty acid greases and animal fats (FFA>20%). In certain cases, it may be desired to subject the feedstock to a refining treatment (e.g., physical refining, chemical refining, etc.) to remove a major part of the non-triglyceride and non-fatty acid components. Physical refining, for example, can remove the FFA, as well as the unsaponifiables and other impurities by steam stripping, thus eliminating the production of soapstock and keeping neutral oil loss to a minimum. However, degumming pretreatments of the crude fats and oils may be required to remove those impurities that darken or otherwise cause a poor-quality product when heated to the temperature required for steam distillation. A degumming process may include treatment of crude oils, with water, salt solutions, enzymes, caustic soda, or diluted acids such as phosphoric, citric or maleic to remove phosphatides, waxes, pro-oxidants and other impurities. The degumming processes convert the phosphatides to hydrated gums, which are insoluble in oil and readily separated as a sludge by settling, filtering or centrifugal action.

The optionally refined oil may contain an unsaturated or substantially unsaturated, liquid or substantially liquid triglyceride part (phase L) and a saturated or substantially saturated, solid or substantially solid triglyceride part (phase S). The solid phase S may thereafter be transformed into linear or substantially linear paraffins as the “bio-naphtha.” More particularly, the refined oil can be fractioned into the phases L and S by a fractional crystallization method that includes a controlled cooling down during which the triglycerides of the complex mixture with substantially saturated acyl-moieties crystallize and precipitate from the mixture forming the phase S, while the triglycerides with substantially unsaturated acyl-moieties remain liquid forming the phase L, both phases being then separated by simple filtration or decantation or centrifugation. In one embodiment, fractionation may be “dry fractionation” or “dry winterization”, which is the removal of solids by controlled crystallization and separation techniques involving the use of solvents or dry processing (sometimes also referred to as dewaxing). The fractionation process has two main stages, the first being the crystallization stage. Crystals grow when the temperature of the molten fat and oil or its solution is lowered, and their solubility at the final or separation temperature determines the triglycerides composition of the crystals formed as well as their mother liquor. The separation process is the second step of fractionation. Several options may be employed, such as vacuum filters, centrifugal separators, conical screen-scroll centrifuges, hydraulic presses, membrane filter presses, or decanters. Fractionation may also been carried out in presence of solvents, such as paraffins, alkyl-acetates, ethers, ketones, alcohols or chlorinated hydrocarbons.

Once obtained, the phase S may converted into linear or substantially linear paraffins as bio-naphtha through known processes, such as thermal decarboxylation (e.g., using a soap feedstock obtained from chemically refining fats and oils), catalytic decarboxylation (e.g., using fatty acid feedstock obtained by physically refining oils and fats), and catalytic hydrodeoxygenation (using triglyceride and/or fatty acid feedstock). Hydrodeoxygenation is often employed and ultimately involves the removal of oxygen atoms from the fats and oils. Hydrodeoxygenation is preferentially done in continuous fixed bed reactors, continuous stirred tank reactors or slurry type reactors containing a solid catalyst. The catalyst may include, for instance, Ni, Mo, Co or mixtures, such as NiW, NiMo, CoMo, NiCOW, NiCoMo, NiMoW and CoMoW oxides or sulphides as catalytic active phase, preferably supported on high surface area carbon, alumina, silica, titania or zirconia or group 10 (Ni, Pt or Pd) or group 11 (Cu or Ag) metals or alloy mixtures supported on high surface area carbon, magnesia, zinc-oxide, spinels (Mg₂Al₂O₄, ZnAl₂O₄), perovskites (BaTiO₃, ZnTiO₃), calcium silicates (e.g., xonotlite), alumina, silica or mixtures of the latter. It is preferred that the support for the catalytic active phase exhibit low acidity, preferable neutral or basic in order to avoid hydro-isomerization reactions that would result in branched paraffins and cracking. Hydrodeoxygenation may be carried out at a temperature from about 200° C. to about 500° C., and in some embodiments, from about 280° C. to about 400° C., under a pressure of from about 1 MPa to about 10 MPa and with a hydrogen to refined oils ratio of from about 100 to about 2000, and in some embodiments, from about 350 to about 1500.

Regardless of the manner in which it is formed, the resulting feedstock containing bio-naphtha may be subjected to a steam cracking process to obtain a bio-aromatic compound (e.g., bio-benzene), which can be used in more or more monomers of the bio-LCP. Steam crackers are complex industrial facilities that can be divided into three main zones, each of which has several types of equipment with very specific functions: (i) the hot zone including: pyrolysis or cracking furnaces, quench exchanger and quench ring, the columns of the hot separation train (ii) the compression zone including: a cracked gas compressor, purification and separation columns, dryers and (iii) the cold zone including: the cold box, de-methanizer, fractionating columns of the cold separation train, the C₂ and C₃ converters, the gasoline hydrostabilization reactor. Hydrocarbon cracking may be carried out in tubular reactors in direct-fired heaters (furnaces). Various tube sizes and configurations can be used, such as coiled tube, U-tube, or straight tube layouts. Each furnace consists of a convection zone in which the waste heat is recovered and a radiant zone in which pyrolysis takes place. The feedstock-steam mixture is preheated in the convection zone to about 530-650° C. or the feedstock is preheated in the convection section and subsequently mixed with dilution steam before it flows over to the radiant zone, where pyrolysis takes place at temperatures varying from 750 to 950° C. The steam/feedstock (the steam/[hydrocarbon feedstock]) weight ratio may be from about 0.2 to about 1.0 kg/kg. For steam cracking furnaces, the severity can be modulated by: temperature, residence time, total pressure and partial pressure of hydrocarbons. Effluent from the pyrolysis furnaces contains unreacted feedstock, olefins (mainly ethylene and propylene), hydrogen, methane, a mixture of C₄ (primarily isobutylene and butadiene), aromatics in the C₆ to C₈ range, ethane, propane, di-olefins (acetylene, methyl acetylene, propadiene), and heavier hydrocarbons that boil in the temperature range of fuel oil. This cracked gas is rapidly quenched to 338-510° C. to stop the pyrolysis reactions, minimize consecutive reactions and to recover the sensible heat in the gas by generating high-pressure steam in parallel transfer-line heat exchangers (TLEs).

The resulting mixed hydrocarbon feed may then be supplied to a “dearomatization unit”, which is a refinery unit for the separation of aromatic hydrocarbons (e.g., bio-benzene). Such dearomatization processes are described in Folkins (2000) Benzene, Ullmann's Encyclopedia of Industrial Chemistry. One particular method to separate aromatic hydrocarbons from a mixture of aromatic and aliphatic hydrocarbons is solvent extraction, such as described in WO 2012/135111, which is incorporated herein by reference thereto. The preferred solvents used in aromatic solvent extraction are sulfolane, tetraethylene glycol and N-methylpyrolidone which are commonly used solvents in commercial aromatics extraction processes. These species are often used in combination with other solvents or other chemicals (sometimes called co-solvents) such as water and/or alcohols. Non-nitrogen containing solvents such as sulfolane are particularly preferred. Solvent extraction of heavy aromatics is described in the art; see e.g. U.S. Pat. No. 5,880,325, which is incorporated herein in its entirety by reference thereto. Alternatively, other known methods than solvent extraction, such as molecular sieve separation or separation based on boiling point, can be applied for the separation of heavy aromatics in a dearomatization process.

Depending on the particular monomer in which the bio-benzene is employed, the subsequent reaction steps may vary. In one embodiment, for instance, hydroxybenzoic acids employed in the polymer (e.g., HBA) may be “bio-hydroxybenzoic acids”, such as those that are derived from “bio-naphtha.” In such embodiments, the bio-benzene may be converted into bio-phenol, which is a reactant employed in the production of bio-hydroxybenzoic acids. In this regard, the bio-benzene may initially be converted into bio-cumene (isopropylbenzene). For example, one suitable process involves an alkylation reaction using propylene and bio-benzene feedstocks. A typical propylene feedstock may be an almost pure polymer grade material or can contain significant amounts of propane, as typically found in refinery-grade propylene. A typical bio-benzene feedstock may contain benzene (99.9 wt.-% min.) and toluene (0.05 wt.-% min). Alkylation reactors may be operated in the vapor phase, liquid-phase or mixed-phase. At the lower temperatures of the liquid phase operation, xylene impurities are not produced and a cumene product of superior quality is produced. The temperature is typically from about 100° C. to about 310° C. and the pressure is typically from about 8 to 50 bar. The alkylation reactor may contain an effective amount of an alkylation catalyst, such as solid acid catalysts (e.g., solid oxide zeolite). Examples are zeolite beta, zeolite X, zeolite Y, mordenite, faujasite, zeolite omega, UZM-8, MCM-22, MCM-36, MCM-49 and MCM-56.

In the alkylation reactor, the bio-benzene is alkylated with the propylene to form bio-cumene (isopropylbenzene). However, some polyisopropyl benzenes, which are mainly di- and tri-substituted propylbenzenes, are also formed. To minimize the production of dialkylated products of benzene, it is generally desired to maintain a molar excess of benzene throughout the reaction zone ranging from about 4:1 to about 16:1, and more preferably about 8:1 of benzene to propylene. A transalkylation reactor may also be used to transalkylate the polyisopropyl benzene produced in the alkylation reactor to form additional cumene. Suitable conditions and catalysts may be the same as described for the alkylation reactor. The alkylator and transalkylator effluents undergo separation operations to separate benzene, cumene product, polyisopropylbenzene, and by-product streams using distillation columns, such as described in U.S. Patent Publication No. 2008/0293986, which is incorporated herein by reference thereto. For example, a first distillation column may be employed that is used to recover excess benzene from the reactor effluents. The benzene column overhead, which is largely benzene, is typically recycled to the alkylator and transalkylator. A second distillation column may be employed to recover the cumene product from the benzene column bottoms. The cumene product is typically the net overhead from the cumene column. A third distillation column may also be employed that is a polyisopropylbenzene column used to recover polyisopropylbenzene recycle stream from the cumene column bottoms. Polyisopropylbenzene is recovered as overhead from the polyisopropylbenzene column and is typically recycled to the transalkylator.

Once formed, the bio-cumene may then be reacted to form bio-phenol using a process known as the “cumene process.” More particularly, the bio-cumene may be initially oxidized to give a cumene hydroperoxide radical. This may occur through oxidation of cumene in an alkaline medium, in which the hydroperoxide product is stable. The bio-cumene may be emulsified in an aqueous alkaline solution such as sodium carbonate, at a pH of 8.5 to 10.5 with an emulsifying agent such as sodium stearate. Oxidation with air or oxygen may occur at mildly elevated temperatures of about 70° C. to about 130° C. The cumene hydroperoxide thereafter undergoes cleaving during which an acid catalyst is added and the hydroperoxide is decomposed to bio-phenol, acetone and other by-products. The acidic catalyst employed can be any acidic material, such as phosphoric acid, sulfuric acid, and SO₂. For example, the cumene hydroperoxide may be treated with dilute sulphuric acid (5 to 25 percent concentration) at a temperature of about 50° C. to about 70° C. After the cleavage is complete, the reaction mixture may be separated and the oil layer distilled to obtain the bio-phenol, acetone, unreacted cumene, alpha-methylstyrene, acetophenone, and tars.

The bio-phenol may thereafter by converted into a bio-hydroxybenzoic acid using, for example, the Kolbe-Schmitt reaction, such as described in U.S. Publication No. 2006/0052632 and U.S. Pat. No. 5,072,036, which are incorporated herein by reference. More particularly, the Kolbe-Schmitt reaction is a carboxylation chemical reaction that proceeds by treating bio-phenol with an alkali metal hydroxide (e.g., potassium hydroxide) to form an alkali metal phenolate (e.g., potassium phenolate), heating the alkali metal phenolate in the presence of carbon dioxide (at elevated or atmospheric pressure), and then optionally treating the product with sulfuric acid. The use of potassium phenolate predominantly primarily results in 4-hydroxybenzoic acid. The temperature at which the alkali metal phenolate is heated typically ranges from about 230° C. to about 450° C. and the carbon dioxide pressure typically ranges from atmospheric pressure to about 6 kg/cm². If desired, the carbon dioxide may be diluted or mixed with gases which are inert to the starting materials and the product under the reaction conditions specified herein. For example, carbon dioxide may be introduced with nitrogen, hydrogen, helium, argon, carbon monoxide, hydrocarbons, etc. The alkali metal phenolate may optionally be reacted with the carbon dioxide in the presence of a substituted phenolate, such as mono-substituted phenolates (e.g., potassium cresolate and potassium phenylphenolate), di-substituted phenolates (e.g., potassium 2,3-, 2,4-, 2,5-, 2,6-, 3,4- and 3,5-xylenolates, dipotassium salt of dihydroxybenzenze), tri-substituted phenolates (e.g., potassium 2,4,6-, 2,3,4-, 2,3,5-, 2,3,6-, 2,4,5-, and 3,4,5-trimethylphenolates), and so forth. When employed, the amount of the substituted phenolate in the reaction system may range from about 0.2 to about 30 equivalents, as calculated in terms of the equivalent of the potassium oxy radical in these compounds based on the equivalent of the starting potassium phenolate. The process may be carried out in an inert reaction medium or it may be performed without using any reaction media. When the process is carried out in an inert reaction medium, examples of such medium include aromatic hydrocarbons, aromatic ethers, aromatic alkanes, aromatic alkenes, aromatic ketones, and hydrogenated products thereof, aliphatic petroleum hydrocarbons, aprotic polar solvents, and higher alcohols.

Besides hydroxybenzoic acids (e.g., HBA), other monomeric constituents may also be derived from a renewable source. For example, in some cases, biphenols may be employed that are “bio-biphenols” that are derived from bio-naphtha using a process such as described above. In such embodiments, the “bio-phenol” referenced in the aforementioned process may be converted into a bio-biphenol. For example, the bio-phenol may be initially alkylated via a Friedel-Crafts alkylation of a hydrocarbon (e.g., isobutene) in the presence of a solid alkylation catalyst (e.g., aluminum phenoxide). Such processes are well known in the art and described, for instance, in U.S. Pat. No. 2,684,389, which is incorporated herein by reference thereto. This reaction may form a bio-dialkylphenol, such as bio-2,4-di-tert-butyl phenol and/or bio-2,6-di-tert-butylphenol according to the following reaction scheme:

C₆H₅OH+2CH₂═C(CH₃)₂→((CH₃)₃C)₂C₆H₃OH

Once formed, the dialkylphenols (at least one of which is a bio-dialkylphenol) are oxidatively coupled to form the bio-biphenol. Such oxidative coupling reactions are described, for instance, in U.S. Patent Publication No. 2003/0050515, which is incorporated herein by reference thereto. In one embodiment, for example, bio-2,4-dialkylphenols and/or bio-2,6-dialkylphenols are oxidatively coupled together, either alone in combination, in the presence of a catalyst (e.g., copper-amino complex) to produce a bio-alkylated biphenol (e.g., bio-2,2′,6,6′-tetraalkyl-4,4′-biphenol). One-step reaction schemes may be employed in some cases. Alternatively, a multi-step reaction scheme may also be employed in which 2,4-dialkylphenol and/or 2,6-dialkylphenol are reacted in a stepwise fashion. For example, the first step may include the synthesis of an alkylated diphenoquinone (e.g., 3,3′,5,5′-tetraalkyl-4,4′-diphenoquinone) using oxygen as an oxidizer to couple 2,6-dialkylphenols. The second step may include the synthesis of an alkylated biphenol (e.g., 2,2′,6,6′-tetraalkyl-4,4′-biphenols) using 3,3′,5,5′-tetraalkyl-4,4′-diphenoquinones as an oxidizer to couple 2,6-dialkylphenols.

If desired, the dialkylphenol(s) (e.g., 2,6-dialkylphenol) may initially be dissolved in a solvent (e.g., methanol, ethanol, isopropanol, butanol, etc.). An excess of the phenol is typically used for the reaction. The temperature of the reaction can be easily and typically within the range of about 30° C. to about 50° C. Typically after approximately 35 to 40% of initial 2,6-dialkylphenols have been converted to the corresponding 3,3′,5,5′-tetraalkyl-4,4′-diphenoquinones, oxygen addition may be stopped and reaction carried out in the absence of oxygen. At this time, the solvent may be removed by distillation for reuse, then the water produced as a by-product of the reaction is removed by distillation at a higher temperature. For the second stage reaction, the temperature may be raised to keep the reaction mixture in a liquid form, typically to about 130° C. to about 160° C. The presence of the catalyst can permit the intermediate 3,3′,5,5′-tetraalkyl-4,4′-diphenoquinones to serve as oxidizing agents for the subsequent coupling of the 2,6-dialkylphenols. After the 3,3′,5,5′-tetraalkyl-4,4′-diphenoquinone is consumed, the reaction mixture generally contains about 70 to 80% 2,2′,6,6′-tetraalkyl-4,4′-biphenols and 20 to 30% 2,6-dialkylphenols. The reaction mixture is then cooled to approximately 60° C. and the solvent is added back to the reaction mixture. In a period of several minutes, the 2,2′,6,6′-tetraalkyl-4,4′-biphenols may precipitate completely and can be removed by filtration or centrifugation, while the 2,6-dialkylphenols and the active catalyst remain in the filtrate. Following the addition of fresh 2,6-dialkylphenol and optional addition of fresh catalyst, these mother-liquors can be re-used to start another cycle of producing of 2,2′,6,6′-tetraalkyl-4,4′-biphenols.

Bio-biphenols (e.g., bio-4,4′-biphenol) can be subsequently produced from the alkylated biphenol (e.g., 2,2′,6,6′-tetra-t-butyl-4,4′-biphenol) through dealkylation. The method of dealkylation may include contacting the alkylated biphenol with an acid that (or optionally a mixture containing an acid and solvent). The dealkylation reaction is usually carried out at a temperature of about 130 to about 170° C. Examples of suitable solvents that can be employed in a reaction mixture include, for instance, hydrocarbons with 7-9 carbons, halogenated hydrocarbons with boiling point about 80 to about 130° C. Examples of suitable acids that can be used in the dealkylation include, for instance, sulfonic acids, such as methanesulfonic acid, sulfuric acid, toluenesulfonic acid, aluminum phenoxides etc.

Regardless of which monomer(s) are derived from bio-naphtha, the type and relative amounts of the monomeric constituents of the liquid crystalline polymer can be selectively controlled to help achieve the desired low melt viscosity. In one embodiment, for example, the composition may employ at least one bio-liquid crystalline polymer (i.e., “first-liquid crystalline polymer”) having a melt viscosity of about 50 Pa-s or less, in some embodiments from about 1 to about 45 Pa-s, in some embodiments from about 2 to about 40 Pa-s, in some embodiments from about 3 to about 35 Pa-s, and in some embodiments, from about 5 to about 20 Pa-s, as determined at a shear rate of 1,000 seconds⁻¹ in accordance with ISO 11443:2021 at a temperature that is 15° C. higher than the melting temperature of the composition.

The first liquid crystalline polymer may contain one or more monomer derived from bio-naphtha. For instance, the polymer repeating units derived from HBA (e.g., bio-HBA), which may constitute from about 40 mol. % to about 80 mol. %, and in some embodiments from about 50 mol. % to about 70 mol. %, and in some embodiments, from about 55 mol. % to about 65 mol. %. Of course, the first liquid crystalline polymer may also contain various other monomers. For example, the first polymer may contain repeating units derived from NDA (e.g., bio-NDA) in an amount of from about 0.1 mol. % to about 20 mol. %, and in some embodiments from about 0.5 mol. % to about 10 mol. %, and in some embodiments, from about 1 mol. % to about 5 mol. %. When employed, the molar ratio of HBA to NDA may be selectively controlled within a specific range to help achieve the desired properties, such as from about 5 to about 50, in some embodiments from about 10 to about 40, and in some embodiments, from about 15 to about 30. The first polymer may also contain aromatic dicarboxylic acid(s) (e.g., IA and/or TA, such as bio-TA and/or bio-IA) in an amount of from about 10 mol. % to about 30 mol. %, and in some embodiments, from about 12 mol. % to about 25 mol. % and/or aromatic diol(s) (e.g., BP and/or HQ, such as bio-BP and/or bio-HQ) in an amount of from about 10 mol. % to about 35 mol. %, and in some embodiments, from about 12 mol. % to about 30 mol. %. When employed, the total amount of TA and IA may be equimolar or in molar excess relative to the total amount of HQ and BP to help achieve the desired properties. In other words, the ratio of the moles of TA and/or IA to the moles of HQ and/or BQ may be about 1 or more, in some embodiments from about 1.05 to about 1.5, and in some embodiments, from about 1.1 to about 1.3. The melt viscosity of the resulting first liquid crystalline polymer may be from about 10 to about 50 Pa-s, in some embodiments from about 15 to about 45 Pa-s, and in some embodiments, from about 20 to about 45 Pa-s, as determined at a shear rate of 1,000 seconds⁻¹ in accordance with ISO 11443:2021 at a temperature that is 15° C. higher than the melting temperature of the composition.

In another embodiment, a second liquid crystalline polymer may be employed, either alone or in combination with the first liquid crystalline polymer. In such embodiments, may also be a bio-LCP in that it contains one or more monomers derived from bio-naphtha. The second liquid crystalline polymer may, for example, contain repeating units derived from HNA (e.g., bio-HNA), which may constitute from about 30 mol. % to about 70 mol. %, and in some embodiments from about 35 mol. % to about 60 mol. %, and in some embodiments, from about 40 mol. % to about 55 mol. %. Of course, the second liquid crystalline polymer may also contain various other monomers. For example, the second polymer may contain repeating units derived from HBA (e.g., bio-HBA) in an amount of from about 0.1 mol. % to about 20 mol. %, and in some embodiments from about 0.5 mol. % to about 10 mol. %, and in some embodiments, from about 1 mol. % to about 5 mol. %. When employed, the molar ratio of HNA to HBA may be selectively controlled within a specific range to help achieve the desired properties, such as from about 5 to about 50, in some embodiments from about 10 to about 40, and in some embodiments, from about 15 to about 30. The polymer may also contain aromatic dicarboxylic acid(s) (e.g., IA and/or TA, such as bio-IA and/or bio-TA) in an amount of from about 10 mol. % to about 35 mol. %, and in some embodiments, from about 15 mol. % to about 30 mol. % and/or aromatic diol(s) (e.g., BP and/or HQ, such as bio-BP and/or bio-HQ) in an amount of from about 10 mol. % to about 35 mol. %, and in some embodiments, from about 15 mol. % to about 30 mol. %. When employed, the total amount of TA and IA may be equimolar or in molar excess relative to the total amount of HQ and BP to help achieve the desired properties. In other words, the ratio of the moles of TA and/or IA to the moles of HQ and/or BQ may be about 1 or more, in some embodiments from about 1.05 to about 1.5, and in some embodiments, from about 1.1 to about 1.3. The melt viscosity of the resulting second liquid crystalline polymer may be from about 1 to about 30 Pa-s, in some embodiments from about 2 to about 20 Pa-s, and in some embodiments, from about 3 to about 10 Pa-s, as determined at a shear rate of 1,000 seconds⁻¹ in accordance with ISO 11443:2021 at a temperature that is 15° C. higher than the melting temperature of the composition.

To help achieve the desired properties of the resulting polymer composition, blends of liquid crystalline polymers may also be employed in the polymer matrix. For example, the polymer composition may contain a blend of the first and second liquid crystalline polymer. In such embodiments, the first liquid crystalline polymer may be employed in the polymer matrix in an amount greater than the second liquid crystalline polymer such that the weight ratio of the first liquid crystalline polymer to the second liquid crystalline polymer may range from about 0.1 to about 20, in some embodiments from about 0.5 to about 20, in some embodiments from about 1 to about 20, in some embodiments from about 1.5 to about 15, and in some embodiments, from about 2 to about 10. The first liquid crystalline polymer may, for example, constitute from about 50 wt. % to about 95 wt. %, in some embodiments from about 60 wt. % to about 90 wt. %, and in some embodiments from about 70 wt. % to about 80 wt. % of the polymer matrix, while the second liquid crystalline polymer may constitute from about 5 wt. % to about 50 wt. %, in some embodiments from about 10 wt. % to about 40 wt. %, and in some embodiments from about 20 wt. % to about 30 wt. % of the polymer matrix. Likewise, the first liquid crystalline polymer may, for example, constitute from about 25 wt. % to about 75 wt. %, in some embodiments from about 30 wt. % to about 70 wt. %, and in some embodiments from about 35 wt. % to about 65 wt. % of the entire polymer composition, while the second liquid crystalline polymer may constitute from about 1 wt. % to about 30 wt. %, in some embodiments from about 2 wt. % to about 25 wt. %, and in some embodiments from about 5 wt. % to about 20 wt. % of the entire polymer composition.

Regardless of the particular constituents, the liquid crystalline polymers (e.g., first and/or second liquid crystalline polymers) may be prepared by initially introducing the aromatic monomer(s) used to form the ester repeating units (e.g., aromatic hydroxycarboxylic acid, aromatic dicarboxylic acid, etc.) and/or other repeating units (e.g., aromatic diol, aromatic amide, aromatic amine, etc.) into a reactor vessel to initiate a polycondensation reaction. The particular conditions and steps employed in such reactions are well known, and may be described in more detail in U.S. Pat. No. 4,161,470 to Calundann; U.S. Pat. No. 5,616,680 to Linstid, III, et al.; U.S. Pat. No. 6,114,492 to Linstid, III, et al.; U.S. Pat. No. 6,514,611 to Shepherd, et al.; and WO 2004/058851 to Waggoner. The vessel employed for the reaction is not especially limited, although it is typically desired to employ one that is commonly used in reactions of high viscosity fluids. Examples of such a reaction vessel may include a stirring tank-type apparatus that has an agitator with a variably-shaped stirring blade, such as an anchor type, multistage type, spiral-ribbon type, screw shaft type, etc., or a modified shape thereof. Further examples of such a reaction vessel may include a mixing apparatus commonly used in resin kneading, such as a kneader, a roll mill, a Banbury mixer, etc.

If desired, the reaction may proceed through the acetylation of the monomers as known the art. This may be accomplished by adding an acetylating agent (e.g., acetic anhydride) to the monomers. Acetylation is generally initiated at temperatures of about 90° C. During the initial stage of the acetylation, reflux may be employed to maintain vapor phase temperature below the point at which acetic acid byproduct and anhydride begin to distill. Temperatures during acetylation typically range from between 90° C. to 150° C., and in some embodiments, from about 110° C. to about 150° C. If reflux is used, the vapor phase temperature typically exceeds the boiling point of acetic acid, but remains low enough to retain residual acetic anhydride. For example, acetic anhydride vaporizes at temperatures of about 140° C. Thus, providing the reactor with a vapor phase reflux at a temperature of from about 110° C. to about 130° C. is particularly desirable. To ensure substantially complete reaction, an excess amount of acetic anhydride may be employed. The amount of excess anhydride will vary depending upon the particular acetylation conditions employed, including the presence or absence of reflux. The use of an excess of from about 1 to about 10 mole percent of acetic anhydride, based on the total moles of reactant hydroxyl groups present is not uncommon.

Acetylation may occur in in a separate reactor vessel, or it may occur in situ within the polymerization reactor vessel. When separate reactor vessels are employed, one or more of the monomers may be introduced to the acetylation reactor and subsequently transferred to the polymerization reactor. Likewise, one or more of the monomers may also be directly introduced to the reactor vessel without undergoing pre-acetylation. In addition to the monomers and optional acetylating agents, other components may also be included within the reaction mixture to help facilitate polymerization. For instance, a catalyst may be optionally employed, such as metal salt catalysts (e.g., magnesium acetate, tin (I) acetate, tetrabutyl titanate, lead acetate, sodium acetate, potassium acetate, etc.) and organic compound catalysts (e.g., N-methylimidazole). Such catalysts are typically used in amounts of from about 50 to about 500 parts per million based on the total weight of the recurring unit precursors. When separate reactors are employed, it is typically desired to apply the catalyst to the acetylation reactor rather than the polymerization reactor, although this is by no means a requirement.

The reaction mixture is generally heated to an elevated temperature within the polymerization reactor vessel to initiate melt polycondensation of the reactants. Polycondensation may occur, for instance, within a temperature range of from about 200° C. to about 400° C. For instance, one suitable technique for forming a liquid crystalline polymer may include charging precursor monomers and acetic anhydride into the reactor, heating the mixture to a temperature of from about 90° C. to about 150° C. to acetylize a hydroxyl group of the monomers (e.g., forming acetoxy), and then increasing the temperature to from about 200° C. to about 400° C. to carry out melt polycondensation. As the final polymerization temperatures are approached, volatile byproducts of the reaction (e.g., acetic acid) may also be removed so that the desired molecular weight may be readily achieved. The reaction mixture is generally subjected to agitation during polymerization to ensure good heat and mass transfer, and in turn, good material homogeneity. The rotational velocity of the agitator may vary during the course of the reaction, but typically ranges from about 10 to about 100 revolutions per minute (“rpm”), and in some embodiments, from about 20 to about 80 rpm. To build molecular weight in the melt, the polymerization reaction may also be conducted under vacuum, the application of which facilitates the removal of volatiles formed during the final stages of polycondensation. The vacuum may be created by the application of a suctional pressure, such as within the range of from about 5 to about 30 pounds per square inch (“psi”), and in some embodiments, from about 10 to about 20 psi.

Generally speaking, the molten polymer is discharged from the reactor at a point in which the desired melt viscosity is achieved. As is known in the art, this may be correlated to the torque of the agitator. For example, after the torque of the agitator reaches a predetermined value, nitrogen may be introduced into the reactor so as to convert reduced pressure conditions to pressurized conditions via atmospheric pressure, thereby discharging the resultant polymer from the reactor, typically through an extrusion orifice fitted with a die of desired configuration, cooled, and collected. Commonly, the melt is discharged through a perforated die to form strands that are taken up in a water bath, pelletized and dried.

Although not always required or desired, the melt polymerized polymer may also be subjected to a subsequent solid-state polymerization method to further increase its molecular weight and achieve the desired melt viscosity. Solid-state polymerization may be conducted in the presence of a gas (e.g., air, inert gas, etc.). Suitable inert gases may include, for instance, include nitrogen, helium, argon, neon, krypton, xenon, etc., as well as combinations thereof. The solid-state polymerization reactor vessel can be of virtually any design that will allow the polymer to be maintained at the desired solid-state polymerization temperature for the desired residence time. Examples of such vessels can be those that have a fixed bed, static bed, moving bed, fluidized bed, etc. The temperature at which solid-state polymerization is performed may vary, but is typically within a range of from about 200° C. to about 400° C. The polymerization time will of course vary based on the temperature and target molecular weight. In most cases, however, the solid-state polymerization time will be from about 2 to about 12 hours, and in some embodiments, from about 4 to about 10 hours.

### B. Optional Components

i. Granular Particulate Filler

If desired, the polymer composition may contain a granular particulate filler that is distributed within the polymer matrix. The granular particulate filler typically constitutes from about 1 to about 100 parts by weight, in some embodiments from about 2 to about 85 parts by weight, in some embodiments from about 20 to about 80 parts by weight, and in some embodiments, from about 5 to about 70 parts by weight per 100 parts by weight of the polymer matrix. For example, the granular particulate filler may constitute from about 5 wt. % to about 50 wt. %, in some embodiments from about 8 wt. % to about 40 wt. %, and in some embodiments, from about 10 wt. % to about 35 wt. % of the polymer composition.

Generally speaking, the granular particles may have a median diameter of from about 0.1 to about 20 micrometers, in some embodiments from about 0.5 to about 18 micrometers, in some embodiments from about 1 to about 15 micrometers, in some embodiments from about 1.5 to about 10 micrometers, and in some embodiments, from about 2 to about 8 micrometers, such as determined using laser diffraction techniques in accordance with ISO 13320:2020 (e.g., with a Horiba LA-960 particle size distribution analyzer) and/or by sedimentation analysis (e.g., Sedigraph 5120). If desired, the particles may also have a high specific surface area, such as from about 1 square meters per gram (m²/g) to about 50 m²/g, in some embodiments from about 1.5 m²/g to about 25 m²/g, and in some embodiments, from about 2 m²/g to about 15 m²/g. Surface area may be determined by the physical gas adsorption (BET) method (nitrogen as the adsorption gas) in accordance with DIN 66131:1993. The moisture content may also be relatively low, such as about 5% or less, in some embodiments about 3% or less, and in some embodiments, from about 0.1 to about 1% as determined in accordance with ISO 787-2:1981 at a temperature of 105° C.

Suitable granular particles may include, for instance, those formed from an inorganic material, such as barium sulfate, calcium sulfate, calcium carbonate, talc, etc. In one embodiment, for instance, the granular particulate filler may include barium sulfate particles. When employed, the median diameter of the barium sulfate particles may, for instance, range from about 1 to about 8 micrometers, in some embodiments from about 1.5 to about 6 micrometers, and in some embodiments, from about 2 to about 5 micrometers, such as determined using laser diffraction techniques in accordance with ISO 13320:2020 (e.g., with a Horiba LA-960 particle size distribution analyzer). The barium sulfate particles may, for example, constitute from about 1 to about 20 parts by weight, in some embodiments from about 2 to about 15 parts by weight, and in some embodiments, from about 3 to about 10 parts by weight per 100 parts by weight of the polymer matrix. For example, the barium sulfate particles may constitute from about 1 wt. % to about 50 wt. %, in some embodiments from about 2 wt. % to about 10 wt. %, and in some embodiments, from about 3 wt. % to about 8 wt. % of the polymer composition. Another suitable type of granular particle may include talc. When employed, the median diameter of the talc particles may, for instance, range from about 3 to about 15 micrometers, in some embodiments from about 4 to about 12 micrometers, and in some embodiments, from about 5 to about 10 micrometers, such as determined by sedimentation analysis (e.g., Sedigraph 5120). The talc particles may, for example, constitute from about 10 to about 60 parts by weight, in some embodiments from about 15 to about 50 parts by weight, and in some embodiments, from about 20 to about 40 parts by weight per 100 parts by weight of the polymer matrix. For example, the talc may constitute from about 5 wt. % to about 40 wt. %, in some embodiments from about 10 wt. % to about 35 wt. %, and in some embodiments, from about 15 wt. % to about 30 wt. % of the polymer composition.

The granulate particulate filler may employ a single type of granular particle (e.g., barium sulfate or talc particles) or a blend of such particles. In certain cases, it may be desired to employ a blend of granular particles to help achieve the desired properties. In one embodiment, for example, a blend of barium sulfate and talc particles is employed. In such embodiments, it is typically desired that weight ratio of the talc to barium sulfate particles ranges from about 1 to about 10, in some embodiments from about 2 to about 8, and in some embodiments, from about 3 to about 6.

ii. Non-Granular Mineral Filler

In addition to any optional granular filler particles (e.g., talc, barium sulfate, etc.), such as described above, other types of mineral fillers may also be employed to help further improve the flow and mechanical properties of the composition. When employed, such non-granular mineral fillers may, for example, constitute from about 1 to about 60 parts by weight, in some embodiments from about 5 to about 50 parts by weight, and in some embodiments, from about 10 to about 40 parts by weight per 100 parts by weight of the polymer matrix. For example, non-granular mineral fillers may constitute from about 1 wt. % to about 40 wt. %, in some embodiments from about 2 wt. % to about 35 wt. %, and in some embodiments, from about 5 wt. % to about 30 wt. % of the polymer composition.

Suitable non-granular mineral fillers may include, for instance, flaked-shaped mineral particles, mineral fibers, etc. Flaked-shaped mineral particles, for example, generally have a relatively high aspect ratio (e.g., average diameter divided by average thickness), such as about 4 or more, in some embodiments about 8 or more, and in some embodiments, from about 10 to about 500. In such embodiments, the average diameter of the particles may, for example, range from about 5 micrometers to about 200 micrometers, in some embodiments from about 8 micrometers to about 150 micrometers, and in some embodiments, from about 10 micrometers to about 100 micrometers. The average thickness may likewise be about 2 micrometers or less, in some embodiments from about 5 nanometers to about 1 micrometer, and in some embodiments, from about 20 nanometers to about 500 nanometers such as determined using laser diffraction techniques in accordance with ISO 13320:2020 (e.g., with a Horiba LA-960 particle size distribution analyzer). Suitable flaked-shaped mineral particles may be formed from a natural and/or synthetic silicate mineral, such as mica, halloysite, kaolinite, illite, montmorillonite, vermiculite, palygorskite, pyrophyllite, calcium silicate, aluminum silicate, wollastonite, etc. Mica is particularly suitable. Any form of mica may generally be employed, including, for instance, muscovite (KAI₂ (AISi₃)O₁₀(OH)₂), biotite (K(Mg,Fe)₃(AISi₃)O₁₀(OH)₂), phlogopite (KMg₃(AISi₃)O₁₀(OH)₂), lepidolite (K(Li,Al)₂₋₃(AISi₃)O₁₀(OH)₂), glauconite (K,Na)(Al,Mg,Fe)₂(Si,Al)₄O₁₀(OH)₂), etc. Muscovite-based mica is particularly suitable for use in the polymer composition. When employed, the flaked-shaped particles (e.g., mica) may constitute from about 10 to about 60 parts by weight, in some embodiments from about 15 to about 50 parts by weight, and in some embodiments, from about 20 to about 40 parts by weight per 100 parts by weight of the polymer matrix. For example, flaked-shaped mineral particles may constitute from about 5 wt. % to about 40 wt. %, in some embodiments from about 10 wt. % to about 35 wt. %, and in some embodiments, from about 15 wt. % to about 30 wt. % of the polymer composition.

Suitable mineral fibers may likewise include those that are derived from silicates, such as neosilicates, sorosilicates, inosilicates (e.g., calcium inosilicates, such as wollastonite; calcium magnesium inosilicates, such as tremolite; calcium magnesium iron inosilicates, such as actinolite; magnesium iron inosilicates, such as anthophyllite; etc.), phyllosilicates (e.g., aluminum phyllosilicates, such as palygorskite), tectosilicates, etc.; sulfates, such as calcium sulfates (e.g., dehydrated or anhydrous gypsum); mineral wools (e.g., rock or slag wool); and so forth. Particularly suitable are fibers having the desired hardness value, including fibers derived from inosilicates, such as wollastonite (Mohs hardness of 4.5 to 5.0). The mineral fibers may have a median diameter of from about 1 to about 35 micrometers, in some embodiments from about 2 to about 20 micrometers, in some embodiments from about 3 to about 15 micrometers, and in some embodiments, from about 7 to about 12 micrometers. The mineral fibers may also have a narrow size distribution. That is, at least about 60% by volume of the fibers, in some embodiments at least about 70% by volume of the fibers, and in some embodiments, at least about 80% by volume of the fibers may have a size within the ranges noted above. In addition to possessing the size characteristics noted above, the mineral fibers may also have a relatively high aspect ratio (average length divided by median diameter) to help further improve the mechanical properties and surface quality of the resulting polymer composition. For example, the mineral fibers may have an aspect ratio of from about 2 to about 100, in some embodiments from about 2 to about 50, in some embodiments from about 3 to about 20, and in some embodiments, from about 4 to about 15. The volume average length of such mineral fibers may, for example, range from about 1 to about 200 micrometers, in some embodiments from about 2 to about 150 micrometers, in some embodiments from about 5 to about 100 micrometers, and in some embodiments, from about 10 to about 50 micrometers. When employed, the mineral fibers (e.g., wollastonite fibers) may constitute from about 10 to about 60 parts by weight, in some embodiments from about 15 to about 50 parts by weight, and in some embodiments, from about 20 to about 40 parts by weight per 100 parts by weight of the polymer matrix. For example, mineral fibers may constitute from about 1 wt. % to about 30 wt. %, in some embodiments from about 2 wt. % to about 20 wt. %, and in some embodiments, from about 5 wt. % to about 15 wt. % of the polymer composition.

iii. Metal Hydroxide

While not always necessary, the liquid crystalline polymer(s) employed in the polymer matrix may be formed by a process that includes melt processing the liquid crystalline polymer(s) in the presence of a metal hydroxide. Without intending to be limited by theory, it is believed that the metal hydroxide can effectively “lose” water under the process conditions (e.g., high temperature), which can assist in melt viscosity reduction and improve the flow properties of the polymer composition. When employed, the metal hydroxide typically constitutes from about 0.1 to about 20 parts by weight, in some embodiments from about 0.3 to about 10 parts by weight, in some embodiments from about 0.5 to about 5 parts by weight, and in some embodiments, from about 0.8 to about 3 parts by weight per 100 parts by weight of the polymer matrix. The metal hydroxide may, for instance, constitute from about 0.01 wt. % to about 5 wt. %, in some embodiments from about 0.05 wt. % to about 2 wt. %, and in some embodiments, from about 0.1 wt. % to about 1 wt. % of the polymer composition.

The metal hydroxide typically has the general formula M(OH)ₐO_{b}, where 0≤a≤3 (e.g., 3) and b=(3−a)/2, where M is a metal, such as a transition metal (e.g., copper), alkali metal (e.g., potassium sodium, etc.), alkaline earth metal (e.g., calcium, magnesium, etc.), post-transition group metal (e.g., aluminum), and so forth. Particularly suitable metals include aluminum and magnesium. Examples of suitable metal hydroxides may include, for instance, copper (II) hydroxide (Cu(OH)₂), potassium hydroxide (KOH), sodium hydroxide (NaOH), magnesium hydroxide (Mg(OH)₂), calcium hydroxide (Ca(OH)₂), aluminum hydroxide (Al(OH)₃), and so forth. The metal hydroxide is typically in the form of particles. In one particular embodiment, for example, the metal hydroxide particles include aluminum hydroxide and optionally exhibit a gibbsite crystal phase. The particles may have a relatively small size, such as a median diameter of from about 50 nanometers to about 3,000 nanometers, in some embodiments from about 100 nanometers to about 2,000 nanometers, and in some embodiments, from about 500 nanometers to about 1,500 nanometers. The term “median” diameter as used herein refers to the “D50” size distribution of the particles, which is the point at which 50% of the particles have a smaller size. The particles may likewise have a D90 size distribution within the ranges noted above. The diameter of particles may be determined using known techniques, such as by ultracentrifuge, laser diffraction, etc. For example, particle size distribution can be determined with laser diffraction according to ISO 13320:2020.

iv. Reinforcing Fibers

If desired, reinforcing fibers may also be employed in the polymer composition, such as polymer fibers, metal fibers, carbonaceous fibers (e.g., graphite, carbide, etc.), inorganic fibers, etc., as well as combinations thereof. Inorganic fibers may be particularly suitable, such as those that are derived from glass; and so forth. Glass fibers may be particularly suitable for use in the present invention, such as those formed from E-glass, A-glass, C-glass, D-glass, AR-glass, R-glass, S1-glass, S2-glass, etc., as well as mixtures thereof. If desired, the reinforcing fibers may be provided with a sizing agent or other coating as is known in the art. The cross-sectional area of the fibers may vary as desired. In some embodiments, for example, the fibers may be generally symmetrical in nature (e.g., square, circular, etc.) such that the aspect ratio of from about 0.8 to about 10, in some embodiments from about 2 to about 8, and in some embodiments, from about 3 to about 5. The aspect ratio is determined by dividing the cross-sectional width of the fibers (i.e., in the direction of the major axis) by the cross-sectional thickness of the fibers (i.e., in the direction of the minor axis). In other embodiments, however, it may be desirable to use fibers that have a relatively flat cross-sectional dimension in that they have an aspect ratio of from about 1.5 to about 10, in some embodiments from about 2 to about 8, and in some embodiments, from about 3 to about 5. The shape of such fibers may be in the form of an ellipse, rectangle, rectangle with one or more rounded corners, etc. Regardless of the particular shape, the cross-sectional width of the fibers may be from about 1 to about 50 micrometers, in some embodiments from about 5 to about 45 micrometers, and in some embodiments, from about 10 to about 35 micrometers. The fibers may also have a thickness of from about 0.5 to about 30 micrometers, in some embodiments from about 1 to about 20 micrometers, and in some embodiments, from about 3 to about 15 micrometers. It should be understood that the cross-sectional thickness and/or width need not be uniform over the entire cross-section. In such circumstances, the cross-sectional width is considered as the largest dimension along the major axis of the fiber and the cross-sectional thickness is considered as the largest dimension along the minor axis. For example, the cross-sectional thickness for an elliptical fiber is the minor diameter of the ellipse. The reinforcing fibers may also have a narrow size distribution. That is, at least about 60% by volume of the fibers, in some embodiments at least about 70% by volume of the fibers, and in some embodiments, at least about 80% by volume of the fibers may have a width and/or thickness within the ranges noted above. The fibers may be endless or chopped fibers, such as those having a length of from about 1 to about 15 millimeters, and in some embodiments, from about 2 to about 6 millimeters. The dimension of the fibers (e.g., length, width, and thickness) may be determined using known optical microscopy techniques.

When employed, the amount of reinforcing fibers may be selectively controlled to achieve the desired combination of flow and good mechanical properties. The reinforcing fibers may, for example, be employed in an amount of from about 1 to about 50 parts, in some embodiments from about 5 to about 40 parts, and in some embodiments, from about 10 to about 30 parts per 100 parts by weight of the polymer matrix. The reinforcing fibers may, for instance, constitute from about 5 wt. % to about 40 wt. %, in some embodiments from about 8 wt. % to about 35 wt. %, and in some embodiments, from about 10 wt. % to about 30 wt. % of the polymer composition.

### V. Flame Retardant System

In addition to the components above, the polymer composition may also contain a flame retardant system that is capable of achieving a desired flammability performance without adversely impact other properties of the composition. When employed, the flame retardant system typically constitutes from about 0.01 to about 50 parts, in some embodiments about 0.1 to about 50 parts, in some embodiments from about 0.5 to about 40 parts, in some embodiments from about 1 to about 30 parts, and in some embodiments, from about 2 to about 20 parts per 100 parts by weight of the polymer matrix. For example, the flame retardant system may constitute from about 0.05 to about 25 wt. %, in some embodiments from about 0.1 wt. % to about 20 wt. %, in some embodiments from about 0.5 wt. % to about 15 wt. %, and in some embodiments, from about 1 wt. % to about 10 wt. % of the composition. The flame retardant system generally includes at least one low-halogen flame retardant. Namely, the halogen (e.g., bromine, chlorine, and/or fluorine) content of such a flame retardant is typically about 1,500 parts per million by weight (“ppm”) or less, in some embodiments about 900 ppm or less, and in some embodiments, about 50 ppm or less. In certain embodiments, the flame retardants are complete free of halogens (i.e., 0 ppm).

A variety of low-halogen flame retardants may be employed in the polymer composition. Suitable low-halogen flame retardants may include, for instance, organophosphorous compounds, such as phosphinate salts of a phosphinic acid and/or diphosphinic acid (e.g., zinc diethylphosphinate, aluminum diethylphosphinate, etc.); mono- and oligomeric phosphoric and phosphonic esters (e.g., tributyl phosphate, triphenyl phosphate, tricresyl phosphate, diphenyl cresyl phosphate, diphenyl octyl phosphate, diphenyl 2-ethylcresyl phosphate, tri(isopropylphenyl) phosphate, resorcinol-bridged oligophosphate, etc.); bisphenol A phosphates (e.g., bisphenol A-bridged oligophosphate or bisphenol A bis(diphenyl phosphate); aryl phosphates, phosphonites, and/or phosphonates; hypophosphorous acid salts, etc.; phosphazenes; red phosphorous; salts of phosphorous acid (e.g., aluminum phosphite, zinc phosphite, aluminum phosphonate, zinc phoshonate, calcium phosphate, aluminum phosphate, zinc phosphate, titanium phosphate, iron phosphate, calcium hydrogenphosphate, calcium hydrogenphosphate dihydrate, magnesium hydrogenphosphate, titanium hydrogenphosphate, zinc hydrogenphosphate, aluminum phosphate, aluminum orthophosphate, aluminum hydrogenphosphate, aluminum dihydrogenphosphate, magnesium dihydrogenphosphate, calcium dihydrogenphosphate, zinc dihydrogenphosphate, zinc dihydrogenphosphate dihydrate, aluminum dihydrogenphosphate, calcium pyrophosphate, calcium dihydrogenpyrophosphate, magnesium pyrophosphate, zinc pyrophosphate aluminum pyrophosphate, etc.); azine phosphate salts (e.g., melamine orthophosphate, melamine pyrophosphate, melamine polyphosphate, piperazine orthophosphate, piperazine pyrophosphate, piperazine polyphosphate, etc.); azine metal phosphate salts (e.g., melamine zinc phosphate, melamine magnesium phosphate, melamine calcium phosphate, bismelamine zincodiphosphate, bismelamine aluminotriphosphate, (melamine)₂Mg(HPO₄)₂, (melamine)₂Ca(HPO₄)₂, (melamine)₃Al(HPO₄)₃, (melamine)₂Mg(P₂O₇), (melamine)₂Ca(P₂O₇), (melamine)₂Zn(P₂O₇), (melamine)₃Al(P₂O₇)_{3/2}, etc.); azine poly(metal phosphates)(e.g., melamine poly(zinc phosphate) and/or melamine poly(magnesium phosphate)); and so forth. Inorganic flame retardant compounds may also be employed, such as inorganic molybdates (e.g., zinc molybdate, calcium molybdate, ammonium octamolybdate, zinc molybdate-magnesium silicate); inorganic borates (e.g., zinc borate); and so forth.

While organophosphorous and/or inorganic compounds are suitable in some cases, it may be desirable in other embodiments to employ low-halogen, polymeric flame retardants. Namely, in addition to helping to improve flame retardancy, such flame retardants can exist in a molten form during melt processing, which allows them to be more uniformly blended within the polymer matrix. This improves dispersion of the components and thereby enhances the overall mechanical properties of the composition without adversely impacting melt viscosity. The present inventor has also discovered that the ability of a low-halogen, polymeric flame retardant to be readily dispersed within the polymer matrix can allow for the use of relatively low concentrations to achieve the desired flame retardant properties. Because it is employed in relatively low concentrations, however, the melt viscosity and mechanical properties of the composition are not adversely impacted. In this regard, low-halogen, polymeric flame retardant(s) may constitute from about 60 wt. % to 100 wt. %, in some embodiments from about 80 wt. % to 100 wt. %, and in some embodiments, from about 90 wt. % to 100 wt. % of the flame retardant system. In certain embodiments, for instance, low-halogen, polymeric flame retardant(s) may constitute from about 0.1 to about 30 parts, in some embodiments from about 0.5 to about 20 parts, in some embodiments from about 1 to about 10 parts, and in some embodiments, from about 2 to about 8 parts per 100 parts by weight of the polymer matrix. For example, low-halogen, polymeric flame retardant(s) may constitute from about 0.01 wt. % to about 20 wt. %, in some embodiments from about 0.05 to about 15 wt. %, in some embodiments from about 0.1 wt. % to about 8 wt. %, in some embodiments from about 0.5 wt. % to about 6 wt. %, and in some embodiments, from about 1 wt. % to about 5 wt. % of the composition.

One particularly suitable low-halogen, polymeric flame retardant is a siloxane polymer. Siloxane polymers, also known as polyorganosiloxanes or polysiloxanes, are polymers having alternate silicon and oxygen atoms in the skeletal structure, and containing silicon-bonded hydrocarbon radicals, the hydrocarbon radicals being attached to the silicon through carbon-silicon linkages. In one embodiment, for instance, the siloxane polymer contains at least one polyorganosiloxane composed of units of the general formula I:

RᵣSiO_{(4-r/2)} (I)

wherein,
R independently of one another, are hydrogen or substituted or unsubstituted hydrocarbon radicals, and
r is 0, 1, 2 or 3, with r typically being within the range from 1.9 to 2.1.

Some examples of suitable radicals R include, for instance, alkyl, aryl, alkylaryl, alkenyl or alkynyl, or cycloalkyl groups, optionally substituted, and which may be interrupted by heteroatoms, i.e., may contain heteroatom(s) in the carbon chains or rings. Suitable alkyl radicals, may include, for instance, methyl, ethyl, n-propyl, isopropyl, n-butyl, isobutyl, tert-butyl, n-pentyl, isopentyl, neopentyl and tert-pentyl radicals, hexyl radicals (e.g., n-hexyl), heptyl radicals (e.g., n-heptyl), octyl radicals (e.g., n-octyl), isooctyl radicals (e.g., 2,2,4-trimethylpentyl radical), nonyl radicals (e.g., n-nonyl), decyl radicals (e.g., n-decyl), dodecyl radicals (e.g., n-dodecyl), octadecyl radicals (e.g., n-octadecyl), and so forth. Likewise, suitable cycloalkyl radicals may include cyclopentyl, cyclohexyl cycloheptyl radicals, methylcyclohexyl radicals, and so forth; suitable aryl radicals may include phenyl, biphenyl, naphthyl, anthryl, and phenanthryl radicals; suitable alkylaryl radicals may include o-, m- or p-tolyl radicals, xylyl radicals, ethylphenyl radicals, and so forth; and suitable alkenyl or alkynyl radicals may include vinyl, 1-propenyl, 1-butenyl, 1-pentenyl, 5-hexenyl, butadienyl, hexadienyl, cyclopentenyl, cyclopentadienyl, cyclohexenyl, ethynyl, propargyl 1-propynyl, and so forth. Examples of substituted hydrocarbon radicals are halogenated alkyl radicals (e.g., 3-chloropropyl, 3,3,3-trifluoropropyl, and perfluorohexylethyl) and halogenated aryl radicals (e.g., p-chlorophenyl and p-chlorobenzyl).

In one particular embodiment, the siloxane polymer includes alkyl radicals (e.g., methyl radicals) bonded to at least 70 mol % of the Si atoms and optionally vinyl and/or phenyl radicals bonded to from 0.001 to 30 mol % of the Si atoms. The siloxane polymer is also preferably composed predominantly of diorganosiloxane units. The end groups of the polyorganosiloxanes may be trialkylsiloxy groups, in particular the trimethylsiloxy radical or the dimethylvinylsiloxy radical. However, it is also possible for one or more of these alkyl groups to have been replaced by hydroxy groups or alkoxy groups, such as methoxy or ethoxy radicals. Particularly suitable examples of the siloxane polymer include diorganopolysiloxane end-capped by trimethylsiloxy groups and composed of from 70 to 100 mol % of dimethylsiloxane units and from 0 to 30 mol % of vinylmethylsiloxane units. Other examples of suitable siloxane polymers are also described in U.S. Pat. No. 7,250,127 to Geck, et al., which is incorporated herein by reference.

The siloxane polymer may also include a reactive functionality on at least a portion of the siloxane monomer units of the polymer, such as one or more of vinyl groups, hydroxyl groups, hydrides, isocyanate groups, epoxy groups, acid groups, halogen atoms, alkoxy groups (e.g., methoxy, ethoxy and propoxy), acyloxy groups (e.g., acetoxy and octanoyloxy), ketoximate groups (e.g., dimethylketoxime, methylketoxime and methylethylketoxime), amino groups (e.g., dimethylamino, diethylamino and butylamino), amido groups (e.g., N-methylacetamide and N-ethylacetamide), acid amido groups, amino-oxy groups, mercapto groups, alkenyloxy groups (e.g., vinyloxy, isopropenyloxy, and 1-ethyl-2-methylvinyloxy), alkoxyalkoxy groups (e.g., methoxyethoxy, ethoxyethoxy and methoxypropoxy), aminoxy groups (e.g., dimethylaminoxy and diethylaminoxy), mercapto groups, etc. While not required, crosslinking agents may be employed in certain embodiments to help enhance the degree of linkage of the organopolysiloxane to the polymer matrix. In this case, suitable crosslinking agents may include peroxides, such as dibenzoyl peroxide, bis(2,4-dichlorobenzoyl) peroxide, dicumyl peroxide, bis-4-methylbenzoyl peroxide, or 2,5-bis(tert-butylperoxy)-2,5-dimethylhexane, and mixtures thereof.

The siloxane polymer may have a high molecular weight, such as a number average molecular weight of about 100,000 grams per mole or more, in some embodiments about 200,000 grams per mole or more, and in some embodiments, from about 500,000 grams per mole to about 2,000,000 grams per mole. The high molecular weight of the siloxane polymer may also be reflected by a high viscosity, such as from about 1×10³ to about 1×10⁸ mPa-s, and in some embodiments, from about 1×10⁵ to about 1×10⁸ mPa-s, as determined at a temperature of about 25° C. Due in part the high viscosity and generally hydrophobic nature, the siloxane polymer can exhibit a reduced tendency to migrate or diffuse to the surface of the polymer composition, thereby further functioning as an “anti-drip” additive to help improve flame retardancy.

If desired, the siloxane polymer may be provided in the form of a silicone formulation that also has a high kinematic viscosity. The kinematic viscosity of the formulation may, for instance, be about 1×10⁵ centistokes or more, in some embodiments from about 5×10⁵ to about 1×10⁸ centistokes, and in some embodiments, from about 1×10⁶ to about 1×10⁷ centistokes, as determined at a temperature of about 25° C. If desired, silica particles (e.g., fumed silica) may also be employed in the silicone formulation in combination with the siloxane polymer to help improve the ability of the polymer to be dispersed within the polymer composition. Such silica particles may, for instance, have a particle size of from about 5 nanometers to about 50 nanometers, a surface area of from about 50 square meters per gram (m²/g) to about 600 m²/g, and/or a density of from about 160 kilogram per cubic meter (kg/m³) to about 190 kg/m³. When employed, the silica particles typically constitute from about 10 wt. % to about 60 wt. %, and in some embodiments, from about 20 to about 40 wt. % of the silicone formulation. Likewise, the siloxane polymer referenced above may constitute from about 60 wt. % to about 90 wt. %, and in some embodiments, from about 60 wt. % to about 80 wt. % of the silicone formulation. The silicone formulation may also contain a halogenated additive, such as one containing boric acid. Among other things, the halogenated additive may permit production of a fully free-flowing pelletized organopolysiloxane material. The additive may also include water (e.g., deionized water or relatively high-purity water) and/or a fatty acid salt. Suitable fatty acid salts may include the salts of a metal, such as Al, Ba, Ca, Cd, Co, Cr, Cu, Fe, Li, Mg, Mn, Ni, Pb, Sn, Sr, or Zn with a fatty acid, such as stearates, palmitates, oleates, linoleates, resinates, laurates, octanoate, ricinoleate, 12-hydroxystearate, naphthenates, tallates, etc. Plasticizers may also be employed in the silicone formulation. Examples of suitable plasticizers may include, for instance, dipolyorganosiloxanes terminated with trimethylsiloxy groups or with hydroxy groups and diphenylsilanediol. The structure of the dipolyorganosiloxanes may be composed of dimethylsiloxane units and/or vinylmethylsiloxane units. The plasticizer typically has a viscosity of about 10,0000 mm²/s or less, and in some embodiments, from about 1 to about 5,000 mm²/s, as determined at a temperature of 25° C.

The silicone formulation may be formed from the components above in a variety of ways, such as by mixing the components in a kneader at a temperature of from about 100° C. to about 250° C., and in some embodiments, from about 120° C. to about 200° C. for a time period of from about 10 minutes to about 12 hours, and in some embodiments, from about 30 minutes to about 6 hours. Once the individual components of the composition have been combined, the formulation may be pelletized using conventional techniques, such as with a perforated plate and rotating knife. The resultant pelletized formulation may have a pellet size (i.e., minimum dimension) of from about 1 to about 100 millimeters, and in some embodiments, from about 2 to about 50 millimeters. For example, the pelletized formulation may have a cylindrical structure with a diameter of from about 3 to about 10 millimeters, and in some embodiments, from about 4 to about 8 millimeters, and a height of from about 2 to about 10 millimeters, and in some embodiments, from about 3 to about 8 millimeters.

As noted above, the flame retardant system and/or the polymer composition itself generally has a relatively low content of halogens (i.e., bromine, fluorine, and/or chlorine), such as about 15,000 parts per million (“ppm”) or less, in some embodiments about 10,000 ppm or less, in some embodiments about 5,000 ppm or less, in some embodiments about 200 ppm or less, and in some embodiments, from about 1 ppm to about 1,500 ppm. Nevertheless, in certain embodiments, halogen-based flame retardants may still be employed as an optional component. Particularly suitable halogen-based flame retardants are fluoropolymers, such as polytetrafluoroethylene (PTFE), fluorinated ethylene polypropylene (FEP) copolymers, perfluoroalkoxy (PFA) resins, polychlorotrifluoroethylene (PCTFE) copolymers, ethylene-chlorotrifluoroethylene (ECTFE) copolymers, ethylene-tetrafluoroethylene (ETFE) copolymers, polyvinylidene fluoride (PVDF), polyvinylfluoride (PVF), and copolymers and blends and other combination thereof. When employed, such halogen-based flame retardants typically constitute at most about 1 wt. % or less, in some embodiments about 0.5 wt. % or less, and in some embodiments, about 0.1 wt. % or less of the polymer composition. In other embodiments, of course, the polymer composition may be generally free of such halogen-based flame retardants.

vi. Other Additives

A wide variety of additional additives can also be included in the polymer composition, such as impact modifiers, lubricants, pigments (e.g., carbon black), antioxidants, stabilizers, surfactants, waxes, flame retardants, nucleating agents (e.g., boron nitride), electrically conductive fillers, and other materials added to enhance properties and processability. Lubricants, for example, may be employed in the polymer composition in an amount from about 0.05 wt. % to about 1.5 wt. %, and in some embodiments, from about 0.1 wt. % to about 0.5 wt. % (by weight) of the polymer composition. Examples of such lubricants include fatty acids esters, the salts thereof, esters, fatty acid amides, organic phosphate esters, and hydrocarbon waxes of the type commonly used as lubricants in the processing of engineering plastic materials, including mixtures thereof. Suitable fatty acids typically have a backbone carbon chain of from about 12 to about 60 carbon atoms, such as myristic acid, palmitic acid, stearic acid, arachic acid, montanic acid, octadecinic acid, parinric acid, and so forth. Suitable esters include fatty acid esters, fatty alcohol esters, wax esters, glycerol esters, glycol esters and complex esters. Fatty acid amides include fatty primary amides, fatty secondary amides, methylene and ethylene bisamides and alkanolamides such as, for example, palmitic acid amide, stearic acid amide, oleic acid amide, N,N′-ethylenebisstearamide and so forth. Also suitable are the metal salts of fatty acids such as calcium stearate, zinc stearate, magnesium stearate, and so forth; hydrocarbon waxes, including paraffin waxes, polyolefin and oxidized polyolefin waxes, and microcrystalline waxes. Particularly suitable lubricants are acids, salts, or amides of stearic acid, such as pentaerythritol tetrastearate, calcium stearate, or N,N′-ethylenebisstearamide.

### II. Formation

The components of the polymer composition may be melt processed or blended together. The components may be supplied separately or in combination to an extruder that includes at least one screw rotatably mounted and received within a barrel (e.g., cylindrical barrel) and may define a feed section and a melting section located downstream from the feed section along the length of the screw. The extruder may be a single screw or twin screw extruder. The speed of the screw may be selected to achieve the desired residence time, shear rate, melt processing temperature, etc. For example, the screw speed may range from about 50 to about 800 revolutions per minute (“rpm”), in some embodiments from about 70 to about 150 rpm, and in some embodiments, from about 80 to about 120 rpm. The apparent shear rate during melt blending may also range from about 100 seconds⁻¹ to about 10,000 seconds⁻¹, in some embodiments from about 500 seconds⁻¹ to about 5000 seconds⁻¹, and in some embodiments, from about 800 seconds⁻¹ to about 1200 seconds⁻¹. The apparent shear rate is equal to 4Q/πR³, where Q is the volumetric flow rate (“m³/s”) of the polymer melt and R is the radius (“m”) of the capillary (e.g., extruder die) through which the melted polymer flows.

### III. Electronic Component

Generally speaking, the polymer composition is well suited for use in a wide variety of thin electronic components, such as electrical connectors, camera modules, etc. Examples of products that may contain such electronic components (e.g., connector, camera module, etc.) may include, for instance, cellular telephones, laptop computers, small portable computers (e.g., ultraportable computers, netbook computers, and tablet computers), wrist-watch devices, pendant devices, headphone and earpiece devices, media players with wireless communications capabilities, handheld computers (also sometimes called personal digital assistants), remote controllers, global positioning system (GPS) devices, handheld gaming devices, battery covers, speakers, camera modules, integrated circuits (e.g., SIM cards), housings for electronic devices, electrical controls, circuit breakers, switches, power electronics, printer parts, etc.

When employed in an electrical connector, for instance, the connector may have a variety of configurations. As an example, the electrical connector can define a plurality of passageways or spaces between opposing walls. The passageways can accommodate contact pins to facilitate a large number of distinct electrical connections with the pins. The electrical connector can also be very compact as a result of the polymer composition from which it is formed. For example, the polymer composition may exhibit excellent flow characteristics for forming very small features required to form the electrical connector described herein while also exhibiting minimal warpage when exposed to heat. In this regard, the walls can have respective widths “w” that are relatively thin, such as about 500 micrometers or less, in some embodiments about 400 micrometers or less, in some embodiments from about 25 micrometers to about 350 micrometers, and in some embodiments from about 50 micrometers to about 300 micrometers.

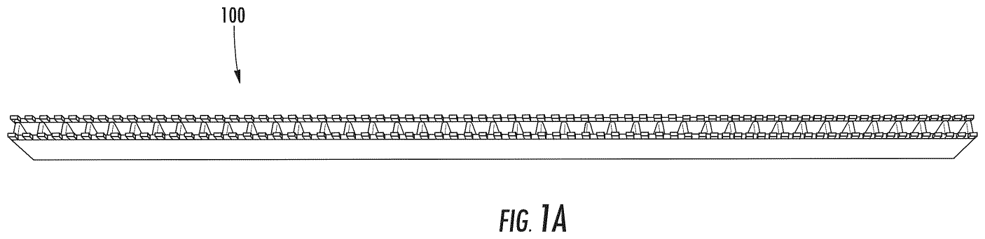

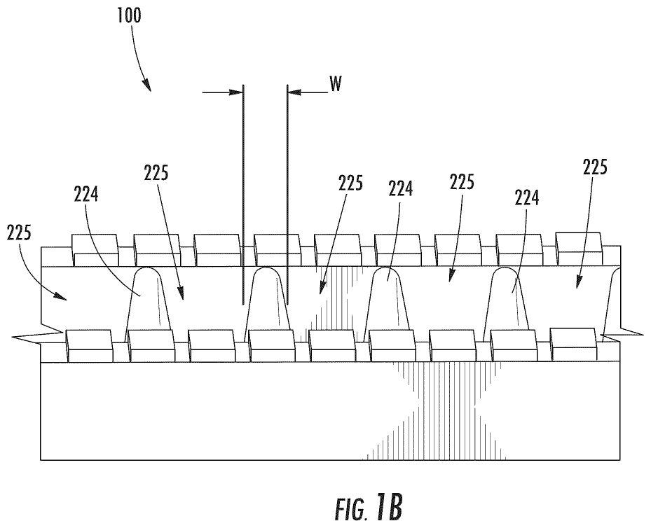

One particularly suitable electrical connector 100 is shown in FIG. 1A according to aspects of the present invention. FIG. 1B depicts an enlarged view of the electrical connector 100 of FIG. 1A. As illustrated, insertion passageways or spaces 225 are defined between opposing walls 224 that can accommodate contact pins to facilitate a large number of distinct electrical connections. The electrical connector 100 can be very compact. More specifically, the walls 224 can have respective widths “w” that are relatively thin, such as within the ranges described above.

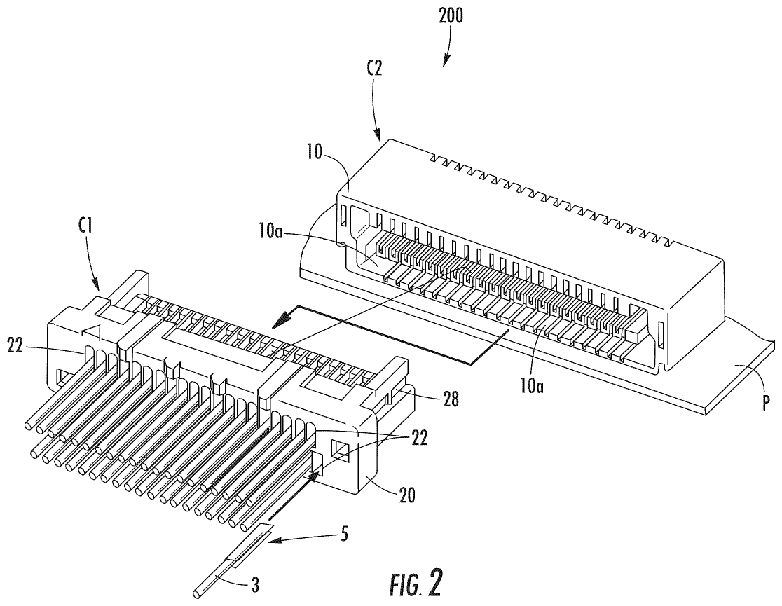

Another embodiment an electrical connector 200 is depicted in FIG. 2. A board-side portion C2 that can be mounted onto the surface of a circuit board P. The connector 200 may also include a wiring material-side portion C1 structured to connect discrete wires 3 to the circuit board P by being coupled to the board-side connector C2. The board-side portion C2 may include a first housing 10 that has a fitting recess 10a into which the wiring material-side connector C1 is fitted and a configuration that is slim and long in the widthwise direction of the housing 10. The wiring material-side portion C1 may likewise include a second housing 20 that is slim and long in the widthwise direction of the housing 20. In the second housing 20, a plurality of terminal-receiving cavities 22 may be provided in parallel in the widthwise direction so as to create a two-tier array including upper and lower terminal-receiving cavities 22. A terminal 5, which is mounted to the distal end of a discrete wire 3, may be received within each of the terminal-receiving cavities 22. If desired, locking portions 28 (engaging portions) may also be provided on the housing 20 that correspond to a connection member (not shown) on the board-side connector C2.

As discussed above, the interior walls of the first housing 10 and/or second housing 20 may be relatively thin (e.g., may have a relatively small width dimension), and can be formed from the polymer composition of the present invention. Of course, the polymer composition may also be employed in any other portion of the connector. Regardless of the portion, the polymer composition may be molded to form the desired shape of the portion (e.g., walls) using known techniques, such as injection molding, low-pressure injection molding, extrusion compression molding, gas injection molding, foam injection molding, low-pressure gas injection molding, low-pressure foam injection molding, gas extrusion compression molding, foam extrusion compression molding, extrusion molding, foam extrusion molding, compression molding, foam compression molding, gas compression molding, etc. For example, an injection molding system may be employed that includes a mold within which the polymer composition may be injected. The time inside the injector may be controlled and optimized so that polymer matrix is not pre-solidified. When the cycle time is reached and the barrel is full for discharge, a piston may be used to inject the composition to the mold cavity. Compression molding systems may also be employed. As with injection molding, the shaping of the polymer composition into the desired article also occurs within a mold. The composition may be placed into the compression mold using any known technique, such as by being picked up by an automated robot arm. The temperature of the mold may be maintained at or above the solidification temperature of the polymer matrix for a desired time period to allow for solidification. The molded product may then be solidified by bringing it to a temperature below that of the melting temperature. The resulting product may be de-molded. The cycle time for each molding process may be adjusted to suit the polymer matrix, to achieve sufficient bonding, and to enhance overall process productivity.

The present invention may be better understood with reference to the following examples and test methods.

### Test Methods

Melt Viscosity: The melt viscosity (Pa-s) may be determined in accordance with ISO 11443:2021 at a shear rate of 400 s⁻¹ or 1,000 s⁻¹ and temperature 15° C. above the melting temperature (e.g., about 325° C.) using a Dynisco LCR7001 capillary rheometer. The rheometer orifice (die) may have a diameter of 1 mm, length of 20 mm, L/D ratio of 20.1, and an entrance angle of 180°. The diameter of the barrel may be 9.55 mm+0.005 mm and the length of the rod may be 233.4 mm.

Melting Temperature: The melting temperature (“Tm”) may be determined by differential scanning calorimetry (“DSC”) as is known in the art. The melting temperature is the differential scanning calorimetry (DSC) peak melt temperature as determined by ISO 11357-3:2018. Under the DSC procedure, samples were heated and cooled at 20° C. per minute as stated in ISO Standard 10350 using DSC measurements conducted on a TA Q2000 Instrument.

Deflection Temperature Under Load (“DTUL”): The deflection under load temperature may be determined in accordance with ISO 75-2:2013 (technically equivalent to ASTM D648). More particularly, a test strip sample having a length of 80 mm, thickness of 10 mm, and width of 4 mm may be subjected to an edgewise three-point bending test in which the specified load (maximum outer fibers stress) was 1.8 Megapascals. The specimen may be lowered into a silicone oil bath where the temperature is raised at 2° C. per minute until it deflects 0.25 mm (0.32 mm for ISO Test No. 75-2:2013).

Tensile Modulus, Tensile Stress, and Tensile Elongation: Tensile properties may be tested according to ISO 527:2019 (technically equivalent to ASTM D638). Modulus and strength measurements may be made on the same test strip sample having a length of 80 mm, thickness of 10 mm, and width of 4 mm. The testing temperature may be 23° C., and the testing speeds may be 1 or 5 mm/min.

Flexural Modulus, Flexural Stress, and Flexural Elongation: Flexural properties may be tested according to ISO 178:2019 (technically equivalent to ASTM D790). This test may be performed on a 64 mm support span. Tests may be run on the center portions of uncut ISO 3167 multi-purpose bars. The testing temperature may be 23° C. and the testing speed may be 2 mm/min.

Charpy Impact Strength: Charpy properties may be tested according to ISO 179-1:2010 (technically equivalent to ASTM D256-10, Method B). This test may be run using a Type 1 specimen size (length of 80 mm, width of 10 mm, and thickness of 4 mm). When testing the notched impact strength, the notch may be a Type A notch (0.25 mm base radius). Specimens may be cut from the center of a multi-purpose bar using a single tooth milling machine. The testing temperature may be 23° C.

Blister-Free Temperature: To test blister resistance, a 127×12.7×0.8 mm test bar may be molded at 5° C. to 10° C. higher than the melting temperature of the polymer resin, as determined by DSC. Ten (10) bars are then placed through a heating chamber of a reflow oven at a predetermined temperature profile, subsequently removed, cooled to ambient conditions, and then inspected for blisters (i.e., surface deformations) that may have formed. The predetermined temperature profile begins at 250° C. and is increased at 10° C. increments until a blister is observed on one or more of the test bars. The highest temperature at which all ten (10) bars exhibit no observable blisters is identified as the “blister free temperature” for a tested material.

Flame Retardancy: The flame retarding efficacy may be determined according to the UL 94 Vertical Burn Test procedure of the “Tests for Flammability of Plastic Materials for Parts in Devices and Appliances” (edition date of Feb. 28, 2023), which is now harmonized with IEC 60707, 60695-11-10 and 60695-11-20 and ISO 9772 and 9773. In the test, two sets of five samples (ten total) may be employed that have a length of 125 mm, width of 13 mm, and a thickness in the desired range (e.g. 0.8 mm, 0.4 mm, or 0.2 mm). The two sets of samples may be conditioned before and after aging. For unaged testing, each thickness is tested after conditioning for 48 hours at 23° C. and 50% relative humidity. For aged testing, five (5) samples of each thickness are tested after conditioning for 168 hours at a temperature of 70° C. and then cooled in a dessicator for at least 4 hours at room temperature. To initiate testing, the sample holder may be positioned so that the sample is held at least 425 mm above a working surface. Cotton batting (e.g., less than 6 mm in thickness) may be placed directly below the clamp to ensure that an area of 50 mm×50 mm is covered. The top 6 mm of the specimen may be placed vertically in the clamp and the holder may be adjusted so that the bottom edge of the specimen is 300±10 mm above the cotton batting. Once in position, the flame may be applied for ten (10) seconds and then removed until flaming stops, at which time the flame may be reapplied for another ten (10) seconds and then removed. If dripping is present, it should fall onto the cotton batting underneath the specimen. When the flaming burn stops, the time is recorded as “t1” and the burner is then reapplied. The time at which flaming burn ends is recorded as “t2.” The time at which the glowing burn ends is recorded as “t3.” The results are then compared to the UL94 flame ratings based on the criteria provided in the table below.

| | | | | # Drips of | | Combustion |
|---|---|---|---|---|---|---|
| | | | | Burning | # Drips of | Up to |
| | Burning Time | | | Specimen | Burning | Holding |
| | of Individual | Total | Burning and | (without | Specimen | Clamp |
| | Specimen (after | Flame | Afterglow | ignition of | (ignition of | (specimens |
| | 1^{st} and 2^{nd} flame | Time(s) | Times | cotton | cotton | completely |
| Rating | applications) (s) | (t1 + t2) | (t2 + t3) | batting) | batting) | burned) |
| V-2 | ≤30 | ≤250 | ≤60 | ≥0 | ≥0 | No |
| V-1 | ≤30 | ≤250 | ≤60 | ≥0 | 0 | No |
| V-0 | ≤10 | ≤50 | ≤30 | ≥0 | 0 | No |

### Examples 1-7

Examples 1-7 are formed from various percentages of a first liquid crystalline polymer (“LCP 1”), second liquid crystalline polymer (“LCP 2”), third liquid crystalline polymer (“LCP 3”), mica, and a first black color concentrate containing 20 wt. % carbon black and 85 wt. % of LCP 3 (“Black 1”). LCP 1 is formed from 48% HNA, 2% HBA, 25% TA, and 25% BP (one or more of which may be derived from bio-naphtha) and has a melting temperature of about 345° C. LCP 1 is formed by charging the aforementioned monomers into a polymerization reactor in combination with an acetylating agent (e.g., acetic anhydride), heating the mixture to acetylize the monomers, and then increasing the temperature to carry out melt polycondensation. The molten polymer is discharged from the reactor at a point in which the extruder torque correlates to a target melt viscosity of about 7 Pa-s (1000 s⁻¹). LCP 2 is formed with the same monomer content as LCP 1, but has a melting temperature of about 346° C. and is formed by subjecting LCP 1 to a solid-stated polymerization process until a target melt viscosity of about 47 Pa-s (1000 s⁻¹) is achieved. LCP 3 is formed from 61.5% HBA, 3% NDA, 16.4% TA, 9.6% BP, and 9.6% HQ (one or more of which may be derived from bio-naphtha), has a melting temperature of about 347° C., and is formed in a manner similar to LCP 1 except that the target melt viscosity is about 39 Pa-s (1000 s⁻¹). The specific formulations are set forth in more detail below in Table 1.

TABLE 1
Formulation

| | Ex. 1 | Ex. 2 | Ex. 3 | Ex. 4 | Ex. 5 | Ex. 6 | Ex. 7 |
|---|---|---|---|---|---|---|---|
| LCP 1 | 7.5 | 15 | 22.5 | 7.5 | — | 7.5 | 15 |
| LCP 2 | — | — | — | 67.5 | 75 | — | — |
| LCP3 | 67.5 | 60 | 52.5 | — | — | 62.5 | 55 |
| Black 1 | — | — | — | — | — | 5 | 5 |
| Mica | 25 | 25 | 25 | 25 | 25 | 25 | 25 |

Samples 1-7 were tested for thermal, mechanical, and flammability. The results are set forth below in Tables 2-6.

TABLE 2
Thermal and Mechanical Properties

| | Ex. 1 | Ex. 2 | Ex. 3 | Ex. 4 | Ex. 5 | Ex. 6 | Ex. 7 |
|---|---|---|---|---|---|---|---|
| Melting Temperature | 331.94 | 343.77 | 313.57 | 338.3 | 343.7 | 322.77 | 316.56 |
| (° C., 1^{st} heat of DSC) | | | | | | | |
| Melt Viscosity at | 16 | 14 | 11 | 25 | 26 | 16.8 | 14.5 |
| 1,000 s⁻¹ (Pa-s) | | | | | | | |
| DTUL (1.8 MPa, ° C.) | 248 | 240 | 230 | 282 | 297 | 181 | 181 |
| Charpy Notched | 6 | 5.2 | 4.4 | 4.1 | 4.0 | 22.0 | 21.0 |
| Strength (kJ/m²) | | | | | | | |
| Charpy Unnotched | 27 | 27 | 21 | 16 | 16 | 26 | 43 |
| Strength (kJ/m²) | | | | | | | |
| Blister Free | 270 | 250 | — | — | — | — | — |
| Temperature (° C.) | | | | | | | |
| Tensile Strength (MPa) | 125 | 125.51 | 121 | 119.08 | 121 | 123 | 128 |
| Tensile Modulus (MPa) | 13284 | 13119 | 12909 | 11411 | 11847 | 7640 | 7687 |
| Tensile Elongation (%) | 2.01 | 2.12 | 2.07 | 2.37 | 2.13 | 4.58 | 4.69 |
| Flexural Strength (MPa) | 152 | 149 | 153 | 173 | 179 | 130 | 130 |
| Flexural Modulus (MPa) | 13219 | 13057 | 12632 | 12105 | 12811 | 8193 | 8110 |
| Flexural Elongation (%) | 2.58 | 2.33 | 2.59 | 3.02 | 2.93 | — | — |

TABLE 3
Unaged Flammability at 0.8 mm

| | Ex. 1 | Ex. 2 | Ex. 3 | Ex. 4 | Ex. 5 | Ex. 6 | Ex. 7 |
|---|---|---|---|---|---|---|---|
| UL 94 Results | V-2 | V-2 | — | — | — | V2 | V2 |
| Total Flame Time | 27.9 | 39.2 | — | — | — | — | — |
| (t1 + t2) (sec) | | | | | | | |
| # burnt over 10 sec | 0 | 0 | — | — | — | 0 | 0 |
| # drips without | 2 | 2 | — | — | — | 4 | 2 |
| ignition of cotton | | | | | | | |
| # drips with | 3 | 2 | — | — | — | 1 | 1 |
| ignition of cotton | | | | | | | |
| # burn to clamp | 0 | 0 | — | — | — | 0 | 0 |

TABLE 4
Aged Flammability at 0.8 mm

| | Ex. 1 | Ex. 2 | Ex. 3 | Ex. 4 | Ex. 5 | Ex. 6 | Ex. 7 |
|---|---|---|---|---|---|---|---|
| UL 94 Results | V-0 | V-0 | — | — | — | V0 | V0 |
| Total Flame Time | 27.9 | 39.2 | — | — | — | — | — |
| (t1 + t2) (sec) | | | | | | | |
| # burnt over 10 sec | 0 | 0 | — | — | — | 0 | 0 |
| # drips without | 5 | 5 | — | — | — | 5 | 4 |
| ignition of cotton | | | | | | | |
| # drips with | 0 | 2 | — | — | — | 0 | 1 |
| ignition of cotton | | | | | | | |
| # burn to clamp | 0 | 0 | — | — | — | 0 | 0 |

TABLE 5
Unaged Flammability at 0.2 mm

| | Ex. 1 | Ex. 2 | Ex. 3 | Ex. 4 | Ex. 5 | Ex. 6 | Ex. 7 |
|---|---|---|---|---|---|---|---|
| UL 94 Results | V-0 | V-2 | — | — | — | V2 | V2 |
| Total Flame Time | 17.1 | 16.9 | — | — | — | — | — |
| (t1 + t2) (sec) | | | | | | | |
| # burnt over 10 sec | 0 | 0 | — | — | — | 0 | 0 |
| # drips without | 2 | 4 | — | — | — | 3 | 2 |
| ignition of cotton | | | | | | | |
| # drips with | 0 | 1 | — | — | — | 2 | 2 |
| ignition of cotton | | | | | | | |
| # burn to clamp | 0 | 0 | — | — | — | 0 | 0 |

TABLE 6
Aged Flammability at 0.2 mm

| | Ex. 1 | Ex. 2 | Ex. 3 | Ex. 4 | Ex. 5 | Ex. 6 | Ex. 7 |
|---|---|---|---|---|---|---|---|
| UL 94 Results | — | — | — | — | — | V2 | V2 |
| Total Flame Time | 24.0 | 24.3 | — | — | — | — | — |
| (t1 + t2) (sec) | | | | | | | |
| # burnt over 10 sec | 0 | 0 | — | — | — | 0 | 0 |
| # drips without | 0 | 2 | — | — | — | 0 | 1 |
| ignition of cotton | | | | | | | |
| # drips with | 3 | 2 | — | — | — | 5 | 4 |
| ignition of cotton | | | | | | | |
| # burn to clamp | 1 | 1 | — | — | — | 0 | 0 |

### Examples 8-14

Examples 8-14 are formed from LCP 1, a fourth liquid crystalline polymer (“LCP 4”), mica, Black 1, barium sulfate (BaSO₄) having an average particle size of 4 micrometers, and a pelletized silicone gum formulation (“Pelletized Silicone”), which is believed to contain trimethylsiloxy-endcapped poly(dimethylsiloxane-co-vinylmethylsiloxane) treated with boric acid/water (100 parts by weight) and fumed silica (30 parts by weight) as described in U.S. Publication No. 2020/0189181 and U.S. Pat. No. 7,250,127. LCP 4 has a melting temperature of about 349° C. and is formed by subjecting LCP 1 to a solid-stated polymerization process until a target melt viscosity of about 67 Pa-s (1000 s⁻¹) is achieved. The specific formulations are set forth in more detail below in Table 7.

TABLE 7
Formulation

| | Ex. 8 | Ex. 9 | Ex. 10 | Ex. 11 | Ex. 12 | Ex. 13 | Ex. 14 |
|---|---|---|---|---|---|---|---|
| LCP 1 | 7.5 | 15 | 15 | 15 | 15 | 15 | 15 |
| LCP 4 | 62.5 | 55 | 55 | 55 | 53 | 53 | 53 |
| Black 1 | 5 | 5 | 5 | 5 | 5 | 5 | 5 |
| Mica | 25 | 25 | 25 | 20 | 25 | 20 | — |
| BaSO₄ | — | — | — | 5 | — | 5 | 30 |
| Pelletized | — | — | — | — | 2 | 2 | — |
| Silicone | | | | | | | |

Samples 8-14 were tested for thermal, mechanical, and flammability. The results are set forth below in Tables 8-12.

TABLE 8
Thermal and Mechanical Properties

| | Ex. 8 | Ex. 9 | Ex. 10 | Ex. 11 | Ex. 12 | Ex. 13 | Ex. 14 |
|---|---|---|---|---|---|---|---|
| Melting Temperature | 329.36 | 319.66 | 346.58 | 346.14 | 343.18 | 350.75 | 338.01 |
| (° C., 1^{st} heat of DSC) | | | | | | | |
| Melt Viscosity at | 23 | 17 | 31 | 18 | 31 | 21 | 9 |
| 1,000 s⁻¹ (Pa-s) | | | | | | | |
| DTUL (1.8 MPa, ° C.) | 254 | 253 | 223 | 221 | 222 | 208 | 182 |
| Charpy Notched | 5.5 | 5 | 4.4 | 4.5 | 4.1 | 4.3 | 3.8 |
| Strength (kJ/m²) | | | | | | | |
| Charpy Unnotched | 29 | 24 | 26 | 27 | 21 | 23 | 17 |
| Strength (kJ/m²) | | | | | | | |
| Blister Free | — | — | 270 | 270 | 260 | 260 | 250 |
| Temperature (° C.) | | | | | | | |
| Tensile Strength (MPa) | 125.4 | 126 | 123 | 121 | 101 | 108 | 109 |
| Tensile Modulus (MPa) | 13562 | 13576 | 12733 | 11639 | 11363 | 10537 | 8094 |
| Tensile Elongation (%) | 1.85 | 1.93 | 2.23 | 2.42 | 1.73 | 2.25 | 2.4 |
| Flexural Strength (MPa) | 159 | 160 | 158 | 153 | 138 | 135 | 128 |
| Flexural Modulus (MPa) | 14439 | 14233 | 12984 | 11859 | 11841 | 10946 | 8169 |
| Flexural Elongation (%) | 2.45 | 2.43 | 2.76 | 2.93 | 2.44 | 2.61 | 3.2 |

TABLE 9
Unaged Flammability at 0.8 mm

| | Ex. 8 | Ex. 9 | Ex. 10 | Ex. 11 | Ex. 12 | Ex. 13 | Ex. 14 |
|---|---|---|---|---|---|---|---|
| UL 94 Results | V-2 | V-0 | V-2 | V-0 | V-0 | V-0 | V-0 |
| Total Flame Time | 30.3 | 27.7 | — | — | — | — | — |
| (t1 + t2) (sec) | | | | | | | |
| # burnt over 10 sec | 0 | 0 | 0 | 0 | 0 | 0 | 0 |
| # drips without | 4 | 5 | 3 | 5 | 0 | 0 | 4 |
| ignition of cotton | | | | | | | |
| # drips with | 1 | 0 | 2 | 0 | 0 | 0 | 0 |
| ignition of cotton | | | | | | | |
| # burn to clamp | 0 | 0 | 0 | 0 | 0 | 0 | 0 |

TABLE 10
Aged Flammability at 0.8 mm

| | Ex. 8 | Ex. 9 | Ex. 10 | Ex. 11 | Ex. 12 | Ex. 13 | Ex. 14 |
|---|---|---|---|---|---|---|---|
| UL 94 Results | V-0 | V-0 | — | — | V-0 | V-0 | V-0 |
| Total Flame Time | 25.6 | 36.9 | — | — | — | — | — |
| (t1 + t2) (sec) | | | | | | | |
| # burnt over 10 sec | 0 | 0 | — | — | 0 | 0 | 0 |
| # drips without | 5 | 5 | — | — | 0 | 0 | 5 |
| ignition of cotton | | | | | | | |
| # drips with | 0 | 0 | — | — | 0 | 0 | 0 |
| ignition of cotton | | | | | | | |
| # burn to clamp | 0 | 0 | — | — | 0 | 0 | 0 |

TABLE 11
Unaged Flammability at 0.2 mm

| | Ex. 8 | Ex. 9 | Ex. 10 | Ex. 11 | Ex. 12 | Ex. 13 | Ex. 14 |
|---|---|---|---|---|---|---|---|
| UL 94 Results | — | — | V-0 | V-2 | V-0 | V-2 | V-2 |
| Total Flame Time | — | — | — | — | — | — | — |
| (t1 + t2) (sec) | | | | | | | |
| # burnt over 10 sec | — | — | 0 | 0 | 0 | 0 | 0 |
| # drips without | — | — | 3 | 5 | 0 | 1 | 4 |
| ignition of cotton | | | | | | | |
| # drips with | — | — | 0 | 2 | 0 | 1 | 1 |
| ignition of cotton | | | | | | | |
| # burn to clamp | — | — | 0 | 0 | 0 | 0 | 0 |

TABLE 12
Aged Flammability at 0.2 mm

| | Ex. 8 | Ex. 9 | Ex. 10 | Ex. 11 | Ex. 12 | Ex. 13 | Ex. 14 |
|---|---|---|---|---|---|---|---|
| UL 94 Results | — | — | V-2 | V-2 | V-2 | V-2 | V-2 |
| Total Flame Time | — | — | — | — | — | — | — |
| (t1 + t2) (sec) | | | | | | | |
| # burnt over 10 sec | — | — | 0 | 0 | 0 | 0 | 0 |
| # drips without | — | — | 0 | 0 | 3 | 3 | 3 |
| ignition of cotton | | | | | | | |
| # drips with | — | — | 5 | 5 | 1 | 1 | 2 |
| ignition of cotton | | | | | | | |
| # burn to clamp | — | — | 0 | 0 | 0 | 0 | 0 |

### Examples 15-17

Examples 15-17 are formed from LCP 1, LCP 4, Black 1, mica, BaSO₄, and Pelletized Silicone. The specific formulations are set forth in more detail below in Table 13.

TABLE 13
Formulation

| | Ex. 15 | Ex. 16 | Ex. 17 |
|---|---|---|---|
| LCP 1 | 15 | 15 | 15 |
| LCP 4 | 53 | 48 | 48 |
| Black 1 | 5 | 5 | 5 |
| Mica | 20 | 20 | 15 |
| BaSO₄ | 5 | 10 | 15 |
| Pelletized Silicone | 2 | 2 | 2 |

Samples 15-17 were tested for thermal, mechanical, and flammability. The results are set forth below in Tables 14-16.

TABLE 14
Thermal and Mechanical Properties

| | Ex. 15 | Ex. 16 | Ex. 17 |
|---|---|---|---|
| Melting Temperature (° C., | 347.25 | 344.2 | 334.51 |
| 1^{st} heat of DSC) | | | |
| Melt Viscosity at 1,000 | 16 | 16 | 13 |
| s⁻¹ (Pa-s) | | | |
| DTUL (1.8 MPa, ° C.) | 247 | 238 | 221 |
| Charpy Notched Strength (kJ/m²) | 4.8 | 3.9 | 3.9 |
| Charpy Unnotched Strength (kJ/m²) | 21 | 20 | 22 |
| Blister Free Temperature | 290 | 290 | 280 |
| (° C.) | | | |
| Tensile Strength (MPa) | 118 | 110 | 117 |
| Tensile Modulus (MPa) | 11390 | 11286 | 10624 |
| Tensile Elongation (%) | 2.07 | 1.9 | 2.31 |
| Flexural Strength (MPa) | 144 | 137 | 142 |
| Flexural Modulus (MPa) | 12532 | 12490 | 11604 |
| Flexural Elongation (%) | 2.52 | 2.21 | 2.69 |

TABLE 15
Unaged Flammability at 0.8 mm

| | Ex. 15 | Ex. 16 | Ex. 17 |
|---|---|---|---|
| UL 94 Results | V-0 | V-0 | V-0 |
| Total Flame Time (t1 + t2) (sec) | — | — | — |
| # burnt over 10 sec | 0 | 0 | 0 |
| # drips without ignition of cotton | 0 | 0 | 0 |
| # drips with ignition of cotton | 0 | 0 | 0 |
| # burn to clamp | 0 | 0 | 0 |

TABLE 16
Aged Flammability at 0.8 mm

| | Ex. 15 | Ex. 16 | Ex. 17 |
|---|---|---|---|
| UL 94 Results | V-0 | V-0 | V-0 |
| Total Flame Time (t1 + t2) (sec) | — | — | — |
| # burnt over 10 sec | 0 | 0 | 0 |
| # drips without ignition of cotton | 0 | 0 | 0 |
| # drips with ignition of cotton | 0 | 0 | 0 |
| # burn to clamp | 0 | 0 | 0 |

### Examples 18-24

Examples 18-24 are formed from LCP 1, LCP 4, Black 1, mica, BaSO₄, and Pelletized Silicone. The specific formulations are set forth in more detail below in Table 17.

TABLE 17
Formulation

| | Ex. 18 | Ex. 19 | Ex. 20 | Ex. 21 | Ex. 22 | Ex. 23 | Ex. 24 |
|---|---|---|---|---|---|---|---|
| LCP 1 | — | — | — | — | 5 | 10 | 15 |
| LCP 4 | 68 | 63 | 63 | 63 | 58 | 53 | 48 |
| Black 1 | 5 | 5 | 5 | 5 | 5 | 5 | 5 |
| Mica | 20 | 20 | 15 | — | 15 | 15 | 15 |
| BaSO₄ | 5 | 10 | 15 | 30 | 15 | 15 | 15 |
| Pelletized | 2 | 2 | 2 | 2 | 2 | 2 | 2 |
| Silicone | | | | | | | |

Samples 18-24 were tested for thermal, mechanical, and flammability. The results are set forth below in Tables 18-20.

TABLE 18
Thermal and Mechanical Properties

| | Ex. 18 | Ex. 19 | Ex. 20 | Ex. 21 | Ex. 22 | Ex. 23 | Ex. 24 |
|---|---|---|---|---|---|---|---|
| Melting Temperature | 337.2 | 334.66 | 335.23 | 333.87 | 347.04 | 342.93 | 338.6 |
| (° C., 1^{st} heat of DSC) | | | | | | | |
| Melt Viscosity at | 33 | 30 | 24 | 16 | 23 | 17.6 | 16.9 |
| 1,000 s⁻¹ (Pa-s) | | | | | | | |
| DTUL (1.8 | 257 | 259 | 263 | — | 237 | 232 | 218 |
| MPa, ° C.) | | | | | | | |
| Charpy Notched | 8 | 5.4 | 10 | 17 | 5.3 | 5 | 4.2 |
| Strength (kJ/m²) | | | | | | | |
| Charpy Unnotched | 30 | 28 | 31 | 48 | 33 | 28 | 25 |
| Strength (kJ/m²) | | | | | | | |
| Blister Free | 260 | 290 | 270 | 270 | — | — | — |
| Temperature (° C.) | | | | | | | |
| Tensile Strength (MPa) | 128 | 126 | 137 | 141 | 116 | 117 | 115 |
| Tensile Modulus (MPa) | 11755 | 12393 | 13140 | 9632 | 10739 | 10656 | 10647 |
| Tensile Elongation (%) | 2.23 | 2.11 | 2.14 | 2.75 | 2.3 | 2.4 | 2.45 |
| Flexural Strength (MPa) | 138 | 141 | 146 | 135 | 136 | 138 | 137 |
| Flexural Modulus (MPa) | 12235 | 13161 | 13651 | 10197 | 11123 | 11399 | 10667 |
| Flexural Elongation (%) | 2.6 | 2.45 | 2.55 | 3.31 | 2.96 | 2.78 | 2.93 |

TABLE 19
Unaged Flammability at 0.8 mm

| | Ex. 18 | Ex. 19 | Ex. 20 | Ex. 21 | Ex. 22 | Ex. 23 | Ex. 24 |
|---|---|---|---|---|---|---|---|
| UL 94 Results | V-0 | V-0 | V-0 | V-0 | V-0 | V-0 | V-0 |
| Total Flame Time | — | — | — | — | — | — | — |
| (t1 + t2) (sec) | | | | | | | |
| # burnt over 10 sec | 0 | 0 | 0 | 0 | 0 | 0 | 0 |
| # drips without | 0 | 0 | 0 | 0 | 0 | 0 | 0 |
| ignition of cotton | | | | | | | |
| # drips with | 0 | 0 | 0 | 0 | 0 | 0 | 0 |
| ignition of cotton | | | | | | | |
| # burn to clamp | 0 | 0 | 0 | 0 | 0 | 0 | 0 |

TABLE 20
Aged Flammability at 0.8 mm

| | Ex. 18 | Ex. 19 | Ex. 20 | Ex. 21 | Ex. 22 | Ex. 23 | Ex. 24 |
|---|---|---|---|---|---|---|---|
| UL 94 Results | V-0 | V-0 | V-0 | V-0 | V-0 | V-0 | V-0 |
| Total Flame Time | — | — | — | — | — | — | — |
| (t1 + t2) (sec) | | | | | | | |
| # burnt over 10 sec | 0 | 0 | 0 | 0 | 0 | 0 | 0 |
| # drips without | 0 | 0 | 0 | 0 | 0 | 0 | 0 |
| ignition of cotton | | | | | | | |
| # drips with | 0 | 0 | 0 | 0 | 0 | 0 | 0 |
| ignition of cotton | | | | | | | |
| # burn to clamp | 0 | 0 | 0 | 0 | 0 | 0 | 0 |

### Examples 25-34

Examples 25-34 are formed from LCP 1, LCP 3, LCP 4, Black 1, a second black color concentrate containing 20 wt. % carbon black and 80 wt. % of LCP 3 (“Black 2”), Aluminum Trihydrate (“ATH”) having an average particle size of 8 micrometers, mica, BaSO₄, and Pelletized Silicone. The specific formulations are set forth in more detail below in Table 21.

TABLE 21
Formulation

| | Ex. | Ex. | Ex. | Ex. | Ex. | Ex. | Ex. | Ex. | Ex. | Ex. |
|---|---|---|---|---|---|---|---|---|---|---|
| | 25 | 26 | 27 | 28 | 29 | 30 | 31 | 32 | 33 | 34 |
| LCP 1 | 15 | 15 | 15 | 15 | — | 15 | 15 | 15 | 15 | — |
| LCP 3 | — | — | — | — | — | 53 | 52.9 | 51.9 | 51.8 | 67.8 |
| LCP 4 | 52.9 | 52.8 | 51.8 | 52.8 | 67.8 | — | — | — | — | — |
| Black 1 | 5 | 5 | 5 | 5 | 5 | — | — | — | — | — |
| Black 2 | — | — | — | — | — | 5 | 5 | 5 | 5 | 5 |
| ATH | 0.1 | 0.2 | 0.2 | 0.2 | 0.2 | — | 0.1 | 0.1 | 0.2 | 0.2 |
| Mica | 20 | 20 | 20 | 25 | 20 | 20 | 20 | 20 | 20 | 20 |
| BaSO₄ | 5 | 5 | 5 | — | 5 | 5 | 5 | 5 | 5 | 5 |
| Pelletized Silicone | 2 | 2 | 3 | 2 | 2 | 2 | 2 | 3 | 3 | 2 |

Samples 25-34 were tested for thermal, mechanical, and flammability. The results are set forth below in Tables 22-24.

TABLE 22
Thermal and Mechanical Properties

| | Ex. | Ex. | Ex. | Ex. | Ex. | Ex. | Ex. | Ex. | Ex. | Ex. |
|---|---|---|---|---|---|---|---|---|---|---|
| | 25 | 26 | 27 | 28 | 29 | 30 | 31 | 32 | 33 | 34 |
| Melting | 342.98 | 343.2 | 343.19 | 345.5 | 347.96 | 345 | 344 | 344 | 343 | 348 |
| Temperature | | | | | | | | | | |
| (° C., 1^{st} heat | | | | | | | | | | |
| of DSC) | | | | | | | | | | |
| Melt | 11 | 9.3 | 10.9 | 13.3 | 14.5 | 16 | 12 | 13 | 10 | 15 |
| Viscosity at | | | | | | | | | | |
| 1,000 s⁻¹ | | | | | | | | | | |
| (Pa-s) | | | | | | | | | | |
| DTUL (1.8 | 240 | 231 | 233 | 225 | 260 | 244 | 240 | 247 | 244 | 262 |
| MPa, ° C.) | | | | | | | | | | |
| Charpy | 3.6 | 3.4 | 3.2 | 3.5 | 4.6 | 4.9 | 4.4 | 3.7 | 3.5 | 4.7 |
| Notched | | | | | | | | | | |
| Strength | | | | | | | | | | |
| (kJ/m²) | | | | | | | | | | |
| Charpy | 23 | 21 | 18 | 20 | 25.3 | 21 | 21 | 19 | 19 | 26 |
| Unnotched | | | | | | | | | | |
| Strength | | | | | | | | | | |
| (kJ/m²) | | | | | | | | | | |
| Blister Free | — | — | — | — | — | 270 | 270 | 270 | 260 | 270 |
| Temperature | | | | | | | | | | |
| (° C.) | | | | | | | | | | |
| Tensile | 109.23 | 106.62 | 98.72 | 100.52 | 113 | 118 | 108 | 106 | 102 | 111 |
| Strength | | | | | | | | | | |
| (MPa) | | | | | | | | | | |
| Tensile | 10660 | 10525 | 10222 | 11331 | 11266 | 11710 | 11421 | 10969 | 10595 | 11424 |
| Modulus | | | | | | | | | | |
| (MPa) | | | | | | | | | | |
| Tensile | 2.16 | 2.16 | 1.97 | 1.75 | 2.09 | 2.2 | 1.89 | 1.95 | 1.9 | 1.95 |
| Elongation | | | | | | | | | | |
| (%) | | | | | | | | | | |
| Flexural | 136 | 136 | 125 | 134 | 133 | 143 | 137 | 134 | 132 | 133 |
| Strength | | | | | | | | | | |
| (MPa) | | | | | | | | | | |
| Flexural | 11014 | 10953 | 10778 | 11716 | 11697 | 12268 | 122025 | 11762 | 11426 | 12121 |
| Modulus | | | | | | | | | | |
| (MPa) | | | | | | | | | | |
| Flexural | 2.7 | 2.7 | 2.24 | 2.27 | 2.47 | 2.53 | 2.34 | 2.38 | 2.33 | 2.32 |
| Elongation | | | | | | | | | | |
| (%) | | | | | | | | | | |

TABLE 23
Unaged Flammability at 0.8 mm

| | Ex. | Ex. | Ex. | Ex. | Ex. | Ex. | Ex. | Ex. | Ex. | Ex. |
|---|---|---|---|---|---|---|---|---|---|---|
| | 25 | 26 | 27 | 28 | 29 | 30 | 31 | 32 | 33 | 34 |
| UL 94 Results | V-0 | V-0 | V-0 | V-0 | V-0 | V-0 | V-0 | V-0 | V-0 | V-0 |
| Total Flame Time | — | — | — | — | — | — | — | — | — | — |
| (t1 + t2) (sec) | | | | | | | | | | |
| # burnt over 10 sec | 0 | 0 | 0 | 0 | 0 | 0 | 0 | 0 | 0 | 0 |
| # drips without | 0 | 0 | 0 | 0 | 0 | 0 | 0 | 0 | 0 | 0 |
| ignition of cotton | | | | | | | | | | |
| # drips with | 0 | 0 | 0 | 0 | 0 | 0 | 0 | 0 | 0 | 0 |
| ignition of cotton | | | | | | | | | | |
| # burn to clamp | 0 | 0 | 0 | 0 | 0 | 0 | 0 | 0 | 0 | 0 |

TABLE 24
Aged Flammability at 0.8 mm

| | Ex. | Ex. | Ex. | Ex. | Ex. | Ex. | Ex. | Ex. | Ex. | Ex. |
|---|---|---|---|---|---|---|---|---|---|---|
| | 25 | 26 | 27 | 28 | 29 | 30 | 31 | 32 | 33 | 34 |
| UL 94 Results | V-0 | V-0 | V-0 | V-0 | V-0 | V-0 | V-0 | V-0 | V-0 | V-0 |
| Total Flame Time | — | — | — | — | — | — | — | — | — | — |
| (t1 + t2) (sec) | | | | | | | | | | |
| # burnt over 10 sec | 0 | 0 | 0 | 0 | 0 | 0 | 0 | 0 | 0 | 0 |
| # drips without | 0 | 0 | 0 | 0 | 0 | 0 | 0 | 0 | 0 | 0 |
| ignition of cotton | | | | | | | | | | |
| # drips with | 0 | 0 | 0 | 0 | 0 | 0 | 0 | 0 | 0 | 0 |
| ignition of cotton | | | | | | | | | | |
| # burn to clamp | 0 | 0 | 0 | 0 | 0 | 0 | 0 | 0 | 0 | 0 |

### Examples 35-42

Examples 35-42 are formed from LCP 1, LCP 3, and LCP 4, Black 1, glass fibers, ATH, mica, BaSO₄, wollastonite (aspect ratio of 13:1) (“Wollastonite”), particle size of 8, talc, and Pelletized Silicone. The specific formulations are set forth in more detail below in Table 25.

TABLE 25
Formulation

| | Ex. 35 | Ex. 36 | Ex. 37 | Ex. 38 | Ex. 39 | Ex. 40 | Ex. 41 | Ex. 42 |
|---|---|---|---|---|---|---|---|---|
| LCP 1 | 15 | 15 | 15 | 15 | 15 | 12 | 13 | 9 |
| LCP3 | — | 51.8 | — | 51.8 | 51.8 | 44.8 | 46.8 | 52.8 |
| LCP 4 | 51.8 | — | 51.8 | — | — | — | — | — |
| Black 1 | 5 | 5 | 5 | 5 | 5 | 5 | 5 | 5 |
| Glass Fibers | — | — | — | — | — | — | — | 15 |
| ATH | 0.2 | 0.2 | 0.2 | 0.2 | 0.2 | 0.2 | 0.2 | 0.2 |
| Mica | 20 | 20 | 20 | 20 | 20 | 20 | 17 | — |
| BaSO4 | 5 | 5 | 5 | 5 | 5 | 5 | 5 | 5 |
| Talc | — | — | — | — | — | — | — | 25 |
| Wollastonite | — | — | — | — | — | 10 | 10 | — |
| Pelletized Silicone | 3 | 3 | 3 | 3 | 3 | 3 | 3 | 3 |

Samples 35-42 were tested for thermal, mechanical, and flammability. The results are set forth below in Tables 26-28.

TABLE 26
Thermal and Mechanical Properties

| | Ex. 35 | Ex. 36 | Ex. 37 | Ex. 38 | Ex. 39 | Ex. 40 | Ex. 41 | Ex. 42 |
|---|---|---|---|---|---|---|---|---|
| Melting Temperature | 344 | 343 | 348 | 344 | 343 | 339 | 340 | 339 |
| (° C., 1^{st} heat of DSC) | | | | | | | | |
| Melt Viscosity at | 30.3 (retest: 8.5) | 23.8 (retest: 8.3) | 15 | 10 | 10 | 18 | 15 | 22 |
| 1,000 s⁻¹ (Pa-s) | | | | | | | | |
| DTUL (1.8 MPa, ° C.) | 224 | 220 | 241 | 237 | 214 | 223 | 226 | — |
| Charpy Notched | 3.5 | 3.2 | 4.3 | 4.1 | 3.1 | 2.2 | 2.2 | 4.2 |
| Strength (kJ/m²) | | | | | | | | |
| Charpy Unnotched | 19 | 17 | 21 | 19 | 18.7 | 9 | 16.3 | 9.7 |
| Strength (kJ/m²) | | | | | | | | |
| Blister Free | 250 | 270 | — | — | 270 | 260 | 270 | 270 |
| Temperature (° C.) | | | | | | | | |
| Tensile Strength (MPa) | 101 | 97 | 114 | 107 | 99 | 88 | 96 | 82 |
| Tensile Modulus (MPa) | 10464 | 10084 | 11407 | 10317 | 9975 | 12393 | 11875 | 12113 |
| Tensile Elongation (%) | 1.99 | 2.07 | 2.1 | 2.13 | 2.06 | 1.03 | 1.3 | 0.89 |
| Flexural Strength (MPa) | 128 | 126 | 136 | 131 | 126 | 129 | 141 | 137 |
| Flexural Modulus (MPa) | 10960 | 10663 | 11851 | 10907 | 10461 | 12891 | 12321 | 12343 |
| Flexural Elongation (%) | 2.34 | 2.4 | 2.43 | 2.39 | 2.37 | 1.61 | 2.11 | 1.53 |

TABLE 27
Unaged Flammability at 0.8 mm

| | Ex. 35 | Ex. 36 | Ex. 37 | Ex. 38 | Ex. 39 | Ex. 40 | Ex. 41 | Ex. 42 |
|---|---|---|---|---|---|---|---|---|
| UL 94 Results | V-0 | V-0 | V-0 | V-0 | — | — | — | — |
| Total Flame Time | — | — | — | — | — | — | — | — |
| (t1 + t2) (sec) | | | | | | | | |
| # burnt over 10 sec | 0 | 0 | 0 | 0 | — | — | — | — |
| # drips without | 0 | 0 | 0 | 0 | — | — | — | — |
| ignition of cotton | | | | | | | | |
| # drips with | 0 | 0 | 0 | 0 | — | — | — | — |
| ignition of cotton | | | | | | | | |
| # burn to clamp | 0 | 0 | 0 | 0 | — | — | — | — |

TABLE 28
Unaged Flammability at 0.8 mm

| | Ex. 35 | Ex. 36 | Ex. 37 | Ex. 38 | Ex. 39 | Ex. 40 | Ex. 41 | Ex. 42 |
|---|---|---|---|---|---|---|---|---|
| UL 94 Results | V-0 | V-0 | V-0 | V-0 | — | — | — | — |
| Total Flame Time | — | — | — | — | — | — | — | — |
| (t1 + t2) (sec) | | | | | | | | |
| # burnt over 10 sec | 0 | 0 | 0 | 0 | — | — | — | — |
| # drips without | 0 | 0 | 0 | 0 | — | — | — | — |
| ignition of cotton | | | | | | | | |
| # drips with | 0 | 0 | 0 | 0 | — | — | — | — |
| ignition of cotton | | | | | | | | |
| # burn to clamp | 0 | 0 | 0 | 0 | — | — | — | — |

### Examples 43-46

Examples 43-46 are formed from LCP 1, LCP 3, Black 1, milled glass fibers, ATH, Mica, BaSO₄, Wollastonite, and Pelletized Silicone. The specific formulations are set forth in more detail below in Table 29.

TABLE 29
Formulation

| | Ex. 43 | Ex. 44 | Ex. 45 | Ex. 46 |
|---|---|---|---|---|
| LCP 1 | 12 | 12 | 13 | 13 |
| LCP 3 | 47.7 | 47.7 | 46.7 | 46.7 |
| Black 1 | 5 | 5 | 5 | 5 |
| Milled Glass Fibers | 10 | 10 | — | — |
| ATH | 0.3 | 0.3 | 0.3 | 0.3 |
| Mica | 17 | 17 | 17 | 17 |
| BaSO₄ | 5 | 5 | 5 | 5 |
| Wollastonite | — | — | 10 | 10 |
| Pelletized Silicone | 3 | 3 | 3 | 3 |

Samples 43-46 were tested for thermal, mechanical, and flammability. The results are set forth below in Tables 30-34.

TABLE 30
Thermal and Mechanical Properties

| | Ex. 43 | Ex. 44 | Ex. 45 | Ex. 46 |
|---|---|---|---|---|
| Melting Temperature | 343 | 344 | 341 | 342 |
| (° C., 1^{st} heat of DSC) | | | | |
| Melt Viscosity at 1,000 | 13 | 7 | 14 | 8 |
| s⁻¹ (Pa-s) | | | | |
| DTUL (1.8 MPa, ° C.) | 226 | 219 | 233 | 239 |
| Charpy Notched | 2.3 | 2.3 | 2 | 2 |
| Strength (kJ/m²) | | | | |
| Charpy Unnotched | 16.9 | 16.6 | 12.8 | 14 |
| Strength (kJ/m²) | | | | |
| Blister Free Temperature | 270 | 260 | 270 | 260 |
| (° C.) | | | | |
| Tensile Strength (MPa) | 99 | 90 | 102 | 89 |
| Tensile Modulus (MPa) | 11519 | 10017 | 12942 | 11248 |
| Tensile Elongation (%) | 1.65 | 1.98 | 1.22 | 1.32 |
| Flexural Strength (MPa) | 133 | 120 | 152 | 133 |
| Flexural Modulus (MPa) | 12000 | 10556 | 13127 | 11684 |
| Flexural Elongation (%) | 2.14 | 2.17 | 2.21 | 1.14 |

TABLE 31
Unaged Flammability at 0.8 mm

| | Ex. 43 | Ex. 44 | Ex. 45 | Ex. 46 |
|---|---|---|---|---|
| UL 94 Results | V-0 | V-0 | V-0 | V-0 |
| Total Flame Time (t1 + t2) (sec) | — | — | — | — |
| # burnt over 10 sec | 0 | 0 | 0 | 0 |
| # drips without ignition of cotton | 0 | 0 | 0 | 0 |
| # drips with ignition of cotton | 0 | 0 | 0 | 0 |
| # burn to clamp | 0 | 0 | 0 | 0 |

TABLE 32
Aged Flammability at 0.8 mm

| | Ex. 43 | Ex. 44 | Ex. 45 | Ex. 46 |
|---|---|---|---|---|
| UL 94 Results | V-0 | V-0 | V-0 | V-0 |
| Total Flame Time (t1 + t2) (sec) | — | — | — | — |
| # burnt over 10 sec | 0 | 0 | 0 | 0 |
| # drips without ignition of cotton | 0 | 0 | 0 | 0 |
| # drips with ignition of cotton | 0 | 0 | 0 | 0 |
| # burn to clamp | 0 | 0 | 0 | 0 |

TABLE 33
Unaged Flammability at 0.2 mm

| | Ex. 43 | Ex. 44 | Ex. 45 | Ex. 46 |
|---|---|---|---|---|
| UL 94 Results | V-0 | V-0 | V-0 | V-0 |
| Total Flame Time (t1 + t2) (sec) | — | — | — | — |
| # burnt over 10 sec | 0 | 0 | 0 | 0 |
| # drips without ignition of cotton | 0 | 2 | 0 | 0 |
| # drips with ignition of cotton | 0 | 0 | 0 | 0 |
| # burn to clamp | 0 | 0 | 0 | 0 |

TABLE 34
Aged Flammability at 0.2 mm

| | Ex. 43 | Ex. 44 | Ex. 45 | Ex. 46 |
|---|---|---|---|---|
| UL 94 Results | V-0 | V-0 | V-0 | V-0 |
| Total Flame Time (t1 + t2) (sec) | — | — | — | — |
| # burnt over 10 sec | 0 | 0 | 0 | 0 |
| # drips without ignition of cotton | 1 | 1 | 0 | 0 |
| # drips with ignition of cotton | 0 | 0 | 0 | 0 |
| # burn to clamp | 0 | 0 | 0 | 0 |

### Examples 47-57

Examples 47-57 are formed from LCP 1, LCP 3, Black 1, glass fibers, ATH, mica, BaSO₄, talc, Wollastonite, and Pelletized Silicone. The specific formulations are set forth in more detail below in Table 35.

TABLE 35
Formulation

| | Ex. | Ex. | Ex. | Ex. | Ex. | Ex. | Ex. | Ex. | Ex. | Ex. | Ex. |
|---|---|---|---|---|---|---|---|---|---|---|---|
| | 47 | 48 | 49 | 50 | 51 | 52 | 53 | 54 | 55 | 56 | 57 |
| LCP 1 | 9 | 9 | 9 | — | 15 | 15 | — | — | — | 9 | — |
| LCP 3 | 37.7 | 32.7 | 37.7 | 46.7 | 51.7 | 51.7 | 66.7 | 59.7 | 59.7 | 41.7 | 50.7 |
| Black 1 | 5 | 5 | 5 | 5 | 5 | 5 | 5 | 5 | 5 | 5 | 5 |
| Glass Fibers | 15 | 20 | 20 | 15 | — | — | — | — | 10 | 15 | 15 |
| ATH | 0.3 | 0.3 | 0.3 | 0.3 | 0.3 | 0.3 | 0.3 | 0.3 | 0.3 | 0.3 | 0.3 |
| Mica | — | — | — | — | 20 | 20 | 20 | 17 | 17 | — | — |
| BaSO₄ | 5 | 5 | 5 | 5 | 5 | 5 | 5 | 5 | 5 | 5 | 5 |
| Talc | 25 | 25 | 20 | 25 | — | — | — | — | — | 21 | 21 |
| Wollastonite | — | — | — | — | — | — | — | 10 | — | — | — |
| Pelletized | 3 | 3 | 3 | 3 | 3 | 3 | 3 | 3 | 3 | 3 | 3 |
| Silicone | | | | | | | | | | | |

Samples 47-57 were tested for thermal, mechanical, and flammability. The results are set forth below in Tables 36-38.

TABLE 36
Thermal and Mechanical Properties

| | Ex. | Ex. | Ex. | Ex. | Ex. | Ex. | Ex. | Ex. | Ex. | Ex. | Ex. |
|---|---|---|---|---|---|---|---|---|---|---|---|
| | 47 | 48 | 49 | 50 | 51 | 52 | 53 | 54 | 55 | 56 | 57 |
| Melting | 342 | 341 | 340 | 350.25 | 344.61 | 340.88 | 348.48 | 349.09 | 348.88 | 329.7 | 344.72 |
| Temperature | | | | | | | | | | | |
| (° C., 1^{st} heat | | | | | | | | | | | |
| of DSC) | | | | | | | | | | | |
| Melt Viscosity | 21 | 23 | 20 | 22 | 9 | 12 | 11 | 18 | 20 | 13 | 14 |
| at 1,000 s⁻¹ | | | | | | | | | | | |
| (Pa-s) | | | | | | | | | | | |
| DTUL (1.8 | 257 | 252 | 260 | 264 | 233 | 224 | 258 | 251 | 255 | 245 | 262 |
| MPa, ° C.) | | | | | | | | | | | |
| Charpy | 4.7 | 4.6 | 4.9 | 4.7 | 2 | 2.7 | 3.1 | 3.7 | 3.2 | 4 | 4.2 |
| Notched | | | | | | | | | | | |
| Strength | | | | | | | | | | | |
| (kJ/m²) | | | | | | | | | | | |
| Charpy | 9.7 | 9.3 | 9.1 | 11 | 17.4 | 24 | 27.5 | 22 | 22.9 | 14 | 16.3 |
| Unnotched | | | | | | | | | | | |
| Strength | | | | | | | | | | | |
| (kJ/m²) | | | | | | | | | | | |
| Blister Free | — | — | — | — | — | — | — | — | — | — | — |
| Temperature | | | | | | | | | | | |
| (° C.) | | | | | | | | | | | |
| Tensile | 86 | 81 | 84 | 79 | 103 | 117 | 122 | 108 | 112 | 94 | 92 |
| Strength | | | | | | | | | | | |
| (MPa) | | | | | | | | | | | |
| Tensile | 12347 | 13721 | 13385 | 12184 | 10237 | 11209 | 13219 | 10327 | 11460 | 11013 | 11907 |
| Modulus | | | | | | | | | | | |
| (MPa) | | | | | | | | | | | |
| Tensile | 0.92 | 0.72 | 0.76 | 0.85 | 1.99 | 2.01 | 1.72 | 2.11 | 2.07 | 1.32 | 1.13 |
| Elongation | | | | | | | | | | | |
| (%) | | | | | | | | | | | |
| Flexural | 130 | 138 | 142 | 127 | 127 | 145 | 153 | 127 | 134 | 137 | 138 |
| Strength | | | | | | | | | | | |
| (MPa) | | | | | | | | | | | |
| Flexural | 12456 | 13876 | 13478 | 12412 | 11049 | 11618 | 13578 | 10989 | 12128 | 10907 | 11786 |
| Modulus | | | | | | | | | | | |
| (MPa) | | | | | | | | | | | |
| Flexural | 1.41 | 1.28 | 1.36 | 1.4 | 2.27 | 2.72 | 2.38 | 2.43 | 2.3 | 1.75 | 1.64 |
| Elongation | | | | | | | | | | | |
| (%) | | | | | | | | | | | |

TABLE 37
Unaged Flammability at 0.8 mm

| | Ex. | Ex. | Ex. | Ex. | Ex. | Ex. | Ex. | Ex. | Ex. | Ex. | Ex. |
|---|---|---|---|---|---|---|---|---|---|---|---|
| | 47 | 48 | 49 | 50 | 51 | 52 | 53 | 54 | 55 | 56 | 57 |
| UL 94 Results | V-0 | V-0 | V-0 | V-0 | — | — | — | — | — | — | — |
| Total Flame Time | — | — | — | — | — | — | — | — | — | — | — |
| (t1 + t2) (sec) | | | | | | | | | | | |
| # burnt over 10 sec | 0 | 0 | 0 | 0 | — | — | — | — | — | — | — |
| # drips without | 0 | 0 | 0 | 0 | — | — | — | — | — | — | — |
| ignition of cotton | | | | | | | | | | | |
| # drips with | 0 | 0 | 0 | 0 | — | — | — | — | — | — | — |
| ignition of cotton | | | | | | | | | | | |
| # burn to clamp | 0 | 0 | 0 | 0 | — | — | — | — | — | — | — |

TABLE 38
Aged Flammability at 0.8 mm

| | Ex. | Ex. | Ex. | Ex. | Ex. | Ex. | Ex. | Ex. | Ex. | Ex. | Ex. |
|---|---|---|---|---|---|---|---|---|---|---|---|
| | 47 | 48 | 49 | 50 | 51 | 52 | 53 | 54 | 55 | 56 | 57 |
| UL 94 Results | V-0 | V-0 | V-0 | V-0 | — | — | — | — | — | — | — |
| Total Flame Time | — | — | — | — | — | — | — | — | — | — | — |
| (t1 + t2) (sec) | | | | | | | | | | | |
| # burnt over 10 sec | 0 | 0 | 0 | 0 | — | — | — | — | — | — | — |
| # drips without | 0 | 0 | 0 | 0 | — | — | — | — | — | — | — |
| ignition of cotton | | | | | | | | | | | |
| # drips with | 0 | 0 | 0 | 0 | — | — | — | — | — | — | — |
| ignition of cotton | | | | | | | | | | | |
| # burn to clamp | 0 | 0 | 0 | 0 | — | — | — | — | — | — | — |

These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed is:

1. A polymer composition comprising a polymer matrix containing a first liquid crystalline polymer that includes one or more monomers derived from bio-naphtha, wherein the polymer composition exhibits a melt viscosity of about 60 Pa-s or less as determined in accordance with ISO 11443:2021 at a shear rate of 1,000 seconds⁻¹ and temperature that is 15° C. higher than the melting temperature of the composition.

2. The polymer composition of claim 1, wherein the bio-content of the first liquid crystalline polymer is from about 5 wt. % to about 100 wt. %.

3. The polymer composition of claim 1, wherein the bio-naphtha is formed from a bio-distillate feedstock that includes a complex mixture of naturally occurring fats and/or oils.

4. The polymer composition of claim 3, wherein the bio-distillate feedstock includes a fat and/or oil derived from cotton, coconut, corn, palm, peanut, linseed, rice, rapeseed, olive, soybean, sunflower, linola, tallow, tall, castor, butter, milk, or a combination thereof.

5. The polymer composition of claim 3, wherein the bio-naphtha is formed by a method that includes fractionating the bio-distillate feedstock into a substantially liquid triglyceride phase L and a saturated or substantially saturated, solid or substantially solid triglyceride phase S, wherein the bio-naphtha is derived from the phase S.

6. The polymer composition of claim 1, wherein the first liquid crystalline polymer contains repeating units derived from 4-hydroxybenzoic acid.

7. The polymer composition of claim 6, wherein the 4-hydroxybenzoic acid is a bio-4-hydroxybenzoic acid derived from bio-naphtha.

8. The polymer composition of claim 7, wherein the bio-4-hydroxybenzoic acid is formed by a process that includes treating a bio-phenol derived from bio-naphtha with an alkali metal hydroxide to form an alkali metal phenolate, and heating the alkali metal phenolate in the presence of carbon dioxide to form the bio-4-hydroxybenzoic acid.

9. The polymer composition of claim 6, wherein the first liquid crystalline polymer contains repeating units derived 4-hydroxybenzoic acid in an amount of from about 40 mol. % to about 80 mol. %.

10. The polymer composition of claim 6, wherein the first liquid crystalline polymer further contains repeating units derived from 2,6-naphthalenedicarboxylic acid.

11. The polymer composition of claim 10, wherein the repeating units derived from 2,6-naphthalenedicarboxylic acid constitute from about 0.1 to about 20 mol. % of the first liquid crystalline polymer.

12. The polymer composition of claim 10, wherein the first liquid crystalline polymer further contains repeating units derived from terephthalic acid, isophthalic acid, hydroquinone, 4,4′-biphenol, or a combination thereof.

13. The polymer composition of claim 1, wherein the polymer matrix further contains a second liquid crystalline polymer having a melting temperature of about 300° C. or more as determined in accordance with ISO 11357-3:2018 and a melt viscosity of about 50 Pa-s or less as determined in accordance with ISO 11443:2021 at a shear rate of 1,000 seconds⁻¹ and temperature that is 15° C. higher than the melting temperature of the second polymer.

14. The polymer composition of claim 1, wherein the polymer matrix constitutes from about 40 wt. % to about 90 wt. % of the polymer composition.

15. The polymer composition of claim 1, wherein the composition contains one or more optional additives.

16. The polymer composition of claim 15, wherein the polymer composition has a sustainable content of from about 10 wt. % to 100 wt. % based on the total weight of the composition.

17. The polymer composition of claim 1, wherein the polymer composition exhibits a deflection temperature under load of about 170° C. or more as measured according to ISO 75-2:2013 at a specified load of 1.8 MPa.

18. The polymer composition of claim 1, wherein the first liquid crystalline polymer has a melting temperature of about 280° C. or more as determined in accordance with ISO 11357-3:2018 and a melt viscosity of about 50 Pa-s or less as determined in accordance with ISO 11443:2021 at a shear rate of 1,000 seconds⁻¹ and temperature that is 15° C. higher than the melting temperature of the first liquid crystalline polymer.

19. The polymer composition of claim 1, wherein the composition exhibits an unaged V-0 rating at a thickness of 0.8 mm when subjected to a vertical burn test procedure in accordance with UL94.

20. The polymer composition of claim 1, wherein the polymer composition contains a flame retardant system distributed within the polymer matrix, wherein the flame retardant system contains a low-halogen flame retardant.

21. The polymer composition of claim 1, wherein the low-halogen flame retardant includes a siloxane polymer.

22. The polymer composition of claim 1, wherein the polymer composition contains a granular particulate filler in an amount of from about 1 to about 100 parts weight per 100 parts of the polymer matrix.

23. The polymer composition of claim 22, wherein the granular particulate filler includes barium sulfate particles, talc, or a combination thereof.

24. The polymer composition of claim 1, wherein the composition further includes mica, reinforcing fibers, or a combination thereof.

25. The polymer composition of claim 1, wherein the polymer composition is formed by a process that includes melt processing the first liquid crystalline polymer in the presence of a metal hydroxide.

26. The polymer composition of claim 1, wherein the blister-free temperature is about 240° C. or more.

27. An electrical connector that comprises opposing walls between which a passageway is defined for receiving a contact pin, wherein at least one of the walls contains the polymer composition of any claim 1.
